(19) United States
(12) Patent Application Publication
(10) Pub. No.: US 2009/0227044 A1
(43) Pub. Date: Sep. 10, 2009

(54) Microchannel Magneto-Immunoassay

(76) Inventors: Dosi Dosev, Davis, CA (US); Vishal Talwar, Davis, CA (US); Mikaela Nichkova, Davis, CA (US); Ian Kennedy, Davis, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 12/162,322

(22) PCT Filed: Jan. 26, 2007
(86) PCT No.: PCT/US07/02138

§ 371 Date: Apr. 6, 2009

Related U.S. Application Data
(60) Provisional application No. 60/762,620, filed on Jan. 26, 2006.

(51) Int. Cl. G01N 33/553; G01N 21/76

(52) U.S. Cl. 436/526; 436/172

(57) ABSTRACT

A single microchannel is combined with external electromagnets for performing a fast immunoassay within a very small volume. Magnetic/luminescent nanoparticles serve as carriers for the antibodies and as internal luminescent standard. The immunoreaction is accelerated by applying alternating magnetic field by means of the external electromagnets, thus inducing oscillation of the particles and achieving better diffusion during the incubation steps. Using the electromagnets the particles are held into the channel for washing and luminescence detection steps. The luminescence of the particles serves as an internal calibration for the assay and helps to avoid experimental error from particle loss.

### CROSS REFERENCE TO RELATED APPLICATIONS

This application claims priority from U.S. Provisional Patent Application Ser. No. 60/762,620, which is hereby incorporated by reference in its entirety for all purposes.

### STATEMENT REGARDING FEDERALLY SPONSORED RESEARCH OR DEVELOPMENT

The U.S. Government has certain rights in this invention pursuant to Grant No. DBI-0102662 awarded by the National Science Foundation, Grant No. 5P42ES04699 awarded by the National Institutes of Health (National Institute of Environmental Health Sciences), and Grant No. 05-35603-16280 awarded by the U.S. Department of Agriculture.

### BACKGROUND OF THE INVENTION

1. Field of the Invention

This invention relates to the fields of chemistry and biology.

2. Description of the Related Art

Fluorescence is a widely used tool in chemistry and biological science. Fluorescent labeling of molecules is a standard technique in biology. The labels are often organic dyes that give rise to the usual problems of broad spectral features, short lifetime, photobleaching, and potential toxicity to cells. A further drawback of fluorescent dye technology is that the conjugation of dye molecules to biological molecules requires a chemistry that generally is unique to each pair of molecules. Alternative labels may be based on lanthanide-derived phosphors. The recent emerging technology of quantum dots has spawned a new era for the development of fluorescent labels using inorganic complexes or particles. These materials offer substantial advantages over organic dyes including larger Stokes shift, longer emission half-life, narrow emission peak and minimal photo-bleaching. However, quantum dot technology still is in its infancy, and is plagued by many problems including difficulties associated with reproducible manufacture, coating, and derivatization of quantum dot materials.

In addition, although the quantum yield of an individual quantum dot is high, the actual fluorescence intensity of each tiny dot is low. Grouping multiple quantum dots into larger particles is one approach for increasing the fluorescence intensity, but this nascent technology still suffers from drawbacks including difficulties in generating and maintaining uniform particle size distributions. Wider application of quantum dot technology therefore has been limited by the difficulties referred to above.

Alternative labels may be based on lanthanide-derived phosphors. Rare-earth metal elements such as europium are known for their unique optical (fluorescent/phosphorescent) properties. When their salts are dissolved in water, their fluorescence is quenched. Thus, many investigators have used europium and other rare-earth chelates to label biological molecules for the sensitive detection of proteins and nucleic acids, to carry out time-resolved fluorometric assays, and as labels in immunoassays. However, this chelation chemistry often is expensive and complex, and so application of rare-earth chelation technology also has been limited to date.

Recently, nanoparticles have received much attention in biology. These particles can have strong fluorescence that exhibits a spectrally sharp emission peak, large Stokes shift, and less quenching influence by other chemicals. Nanoparticles such as Eu₂O₃ particles also have been recognized as offering tremendous potential in obtaining large enhancement of emission intensity. However, Eu₂O₃ and other nanoparticles are easily dissolved by acid during activation and conjugation, thereby losing their desirable properties. In addition, nanoparticles lack reactive groups that allow them to be easily derivatized and linked to analytes and other reagents, thus increasing the difficulty associated with using nanoparticles as labeling reagents for the study of biological and other molecules.

Silica and alumina surfaces have wide-ranging surface reactivities; in particular, silica can be used as a cap to keep europium oxide from dissolving in acid in the conjugation process. However, coating with silica and alumina may increase the particle size, thereby compromising the advantageous properties of nanoparticles that render them suitable as labeling reagents.

Magnetic beads are another type of particle traditionally used in biochemical and clinical analysis for magnetic separation. Usually, they consist of a magnetic core covered by a polymer shell having a functionally modified surface. Particles having magnetic properties and light emitting properties provide additional benefits such as, e.g., permitting optimized biochemical protocols to be developed useful for both analyte detection and analyte separation or purification. U.S. Pat. No. 6,773,812 describes particles having magnetic and light emitting properties, but the light-emitting properties of those particles are derived from conventional dyes such as fluorescent dyes and so suffer from the associated disadvantages of photobleaching, small Stokes shifts, and short lifetimes.

A large variety of nanoparticles with different properties have been subject to intense research focused on their synthesis, characterization and application in biochemistry [1]. Fluorescent nanoparticles have been demonstrated as promising alternatives to widely used organic fluorescent dyes [2]. Quantum dots [3,4], dye-doped silica [5], chelate-doped polystyrene [6] and lanthanide oxides [7,8] each offer unique advantages and find different applications as fluorescent labels in biotechnology such as cell staining, molecular recognition, immunoassays [3, 9-13], visualization of DNA and protein microarrays [14, 15].

Core/shell structured magnetic nanoparticles are currently of interest in a wide variety of applications. For example, Fe/Au core/shell structured nanoparticles [16, 17], due to the possibility of remote magnetic manipulation [18], may be used in biological applications as magnetic resonance imaging (MRI) agents [19, 20], cell tagging and sorting [21] and targeted drug delivery [22] (for reviews see [23, 24]).

The combination of magnetic and fluorescent properties is a new powerful tool allowing manipulation by magnetic fields and visualization/detection by fluorescence. There are commercially available tools and automatic instruments for manipulating magnetic particles and for detecting a variety of fluorescent labels over the entire visible spectrum.

Recently, several groups reported the successful synthesis of particles that possess both fluorescent and magnetic properties. In most of these works, colloidal solution techniques were employed. Lu et al [25] and Levy et al [26] coated iron oxide particles with silica shells containing organic dyes which provided the fluorescent properties of the nanoparticles. Sahoo et al [27] employed covalent binding of organic dyes on the surface of magnetite particles. Using covalent binding, Wang et al [28] formed a fluorescent shell of quantum dots on polymer-coated iron oxide beads, while Mulvaney et al [29] incorporated organic dyes and quantum dots into the polystyrene shell of magnetic beads. Lu et al [30] formed a shell of up-converting phosphor (ytterbium and erbium co-doped sodium yttrium fluoride) on an iron oxide core that made use of the luminescent properties of the lanthanide ions. In most of the cases, the synthesis of particles with magnetic and fluorescent properties is complicated and expensive. For example, the beads of Wang et al [28] are expensive, toxic (due to the use of quantum dots), are less mechanically stable, have shorter lifetimes, and poorer multiplexing than the particle described in conjunction with the present invention. The need for up-converting as described in Lu et at [30] requires more than one excitation source. An additional drawback is that organic dyes have broad emission spectra and poor photostability. An efficient and low-cost method for synthesis of magnetic/fluorescent particles would be highly beneficial for applications that demand significant amounts of reagents and are economical—environmental monitoring for bioterror agents is a good example. A low-cost synthesis route would allow improvements in biotechnologies and facilitate the creation of new widely applicable biochemical protocols.

A great deal of effort has gone in the development of immunodiagnostics for detecting plant disease and pathogens. For example, nanotechnology has been used to provide rapid detection of single bacterial cells. Fluorophore-doped silica nanoparticles were chemically functionalized for the attachment of antibodies against E. coli. A solution of nanoparticles was mixed with a solution of bacteria, then centrifuged to separate bound and unbound antibodies; the fluorescence of the bound antibody fraction was measured. Zhao [31]. Although sensitive, this method is somewhat slow due to the centrifugation step. A number of other schemes, e.g., as demonstrated by Muhammad-Tahir [32], Lagally [33], Vo-Dinh [34], Dill [35], Sapsford [36], Taitt [37], and Weeks [38], have been advanced over the years to provide portable or disposable, miniaturized analytical systems for the detection of pathogens.

Despite these efforts, novel biosensors are still needed to identify multiple toxins and pathogens in individual samples within a complex background matrix. Increased sensitivity is needed to eliminate the need for enrichment of the pathogens using culture methods and to detect the low levels of toxin that can cause disease. Faster real-time assays will enable more samples to be analyzed and timely results obtained before products enter commerce, thereby ensuring statistically more reliable monitoring of the integrity of the food supply.

Foodborne illness, commonly referred to as food poisoning, results from consumption of food contaminated with various materials including pathogenic bacteria, toxins, viruses, prions or parasites. Most food-borne pathogens have animal reservoirs from which they can infect humans. However, recent outbreaks of a variety of pathogens have been associated with fresh produce or water. Beuchat [39]. Outbreaks on fresh produce may pose a more serious food safety problem since these items are not routinely cooked. Illness also can be caused by exotoxins that are excreted by the bacterium as they grow. Thus, exotoxins are capable of causing illness even when the microbes have been killed. Examples include toxins produced by Clostridium botuliiium, Clostridium perfringens, and Staphylococcus aureus. Recently, prions have been shown as a new form of food-borne pathogen with the discovery that Bovine Spongiform Encephalopathy can be transmitted to humans via contaminated meat, resulting in a variant form of Creutzfeldt-Jakob Disease, a 100% fatal neurological disease. Food borne infections cause millions of illnesses and thousands of deaths every year in the United States. Recent estimates suggest that there are 76 million illnesses caused by food-borne diseases resulting in 325,000 hospitalizations and 5,000 deaths in the United States. Mead [40].

Superimposed on these natural causes of food poisoning, we now face the daunting challenge of intentional adulteration of foods. Moon [41]. Along with the bacterial toxins mentioned above, potent plant-derived toxins are candidates for biological warfare and terrorism Two examples are ricin and abrin. Ricin is widely available, easily produced, and derived from the beans of the castor plant (e.g., Ricinus communis Mirarchi [42]), More toxic than ricin, abrin is a glycoprotein found in the precatory bean (Abrus precatorius). Budavari [43].

Identification of these agents is difficult and time-consuming. For example pathogen detection often involves cell culture and sophisticated chemical identification, along with the need for dedicated laboratories and large reference collections. Toxin detection may require a bioassay or polymerase chain reaction (PCR) method that can take 1-2 days. Arnon [44]. Alternatively, toxins can undergo complex extraction steps followed by some form of chromatography and mass spectrometry for identification. Wannemacher [45]. Identification of prions is even more difficult, necessitating an immunohistochemical and biochemical analysis of the sample. All of these approaches are time-consuming, requiring from days to weeks for a result. By the time such lengthy analyses have been completed, the product has moved into commerce. Thus, the needs of quality control technician in the field and the laboratory scientist for testing the integrity of our food supply are several-fold: (1) fast turn around; (2) multi analyte capability; (3) specificity to particular compounds without false positives; (4) high sensitivity.

In addition to semiconductor quantum dots, simple inorganic phosphors such as Eu₂O₃ have been recognized as offering potential in obtaining high emission intensity. Nogami [46]; Patra [47].

Europium containing nanoparticles have been used previously as labels for time-resolved bioassays. Harma et al. [48] entrapped chelated europium in polystyrene particles that had diameters ranging from 100 to about 400 nm. Carboxyl groups on the surface of the polymer particles were used in the conjugation to biomolecules. Although this scheme was employed successfully, we have devised several simpler methods for using europium oxide nanoparticles directly, without the need for chelation or entrapment in another medium. We found that natural or untreated Eu₂O₃ particles are insoluble in water but are easily dissolved by acid during activation and conjugation, losing their desirable optical properties. Coating the particles by silanization protected the particle from being dissolved by acid, and provided useful functional groups for biological conjugation. Feng [49]. Passive absorption of proteins is an alternative for surface functionalization that we have recently explored. Dosev [50].

The present invention addresses these and other limitations of the prior art by providing improved nanoparticle compositions and methods of use. Derivatized nanoparticle compositions of the present invention retain the optical properties of the native particles and enable the efficient and low-cost use of these nanoparticles to label and optionally separate or purify biological and other materials.

### SUMMARY OF THE INVENTION

The present invention is defined by the following claims, and nothing in this section should be taken as a limitation on those claims. Disclosed herein is an assay comprising magnetic/luminescent nanoparticles. The luminescence of the particles serves as an internal calibration for the assay and helps to avoid experimental error from particle loss. Also disclosed is a microchannel in which the assay may be performed.

In one aspect, the present invention consists of using a single microchannel combined with external electromagnets for performing a fast immunoassay within a very small volume. Magnetic/luminescent nanoparticles provide an internal luminescent standard. According to another aspect of the present invention, a binding reaction is accelerated by applying an alternating magnetic field by alternatingly energizing a plurality of electromagnets external to a microchannel, thus inducing oscillation and/or agitation of the particles and achieving better diffusion during incubation. Using the electromagnets, the particles are held in the channel for washing and luminescence detection steps. The luminescence of the particles serves as an internal calibration for the assay and helps to avoid experimental error from particle loss.

Also described herein is the synthesis and the properties of magnetic/luminescent core/shell particles useful in conjunction with the above-described methods, including magnetic cores of iron oxide doped with cobalt and neodymium (Nd:Co:Fe₂O₃) that are encapsulated in luminescent shells of europium-doped gadolinium oxide (Eu:Gd₂O₃). Cobalt [51] and neodymium [52] were shown to improve the magnetic properties of iron oxides. In addition, doping of Eu ions into the Gd₂O₃ matrix gives unique luminescent properties [53, 54]. The methods described herein employ flame spray pyrolysis as a cost-effective, high throughput and versatile synthesis method, allowing a variety of doped materials to be obtained, such as described in U.S. patent application Ser. Nos. 10/393,702 and 10/576,776, each of which is hereby incorporated by reference hereto for all purposes.

Various particle types that may be used in conjunction with the inventive methods described herein are detailed. In one aspect of the invention a silica glass nanoparticle is co-doped with a rare earth element and another metal element. In another aspect, the invention uses nanoparticles having a magnetic oxide core and a shell comprising a rare earth element and optionally another metal element. In one aspect, the particles useful in conjunction with the methods of the present invention are prepared using gas-phase combustion and/or pyrolysis synthesis. These types of particles provide the additional advantage of absorbing and emitting light at multiple wavelengths further expanding the use of these particles as labels in, e.g., multiplexed applications.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a schematic depiction of one embodiment of the main stages of an assay, e.g., an immunoassay, with an internal luminescent standard, based on magnetic/luminescent particles.

FIGS. 2a-2i are schematic illustrations depicting one embodiment of an assay as described herein performed in a microchannel.

FIG. 3a shows an Eu₂O₃ excitation spectrum monitored at 612 nm.

FIG. 3b shows an Eu₂O₃ emission spectrum excited at 466 nm (full line bare particles without functionalization; dashed line with silence functionalization).

FIG. 4 illustrates detection of atrazine with Eu₂O₃ nanoparticle labels in an immunoassay.

FIG. 5 is a schematic diagram of the forces acting on a particle.

FIG. 6 is a photograph of one embodiment of an apparatus for forming a microchannel.

FIGS. 7a-7c illustrate magnetic separation of magnetic particles.

FIG. 8 is a schematic diagram of sandwich type immunoassay.

FIG. 9 is a schematic of one embodiment of an apparatus for flame synthesis of nanoparticles.

FIG. 10 is a schematic of a pneumatic nebulizer and optional co-flow jacket used in conjunction with the apparatus illustrated in FIG. 9.

FIG. 11 is a schematic of an apparatus for functionalizing aerosolized nanoparticles.

FIG. 12a is a transmission electron micrograph (TEM) of pure Eu₂O₃ nanoparticles.

FIG. 12b shows fluorescence emission spectra for pure Eu₂O₃ nanoparticles (monoclinic phase) excited at 466 nm showing short fluorescence lifetime.

FIG. 13a is a TEM of Eu:Y₂O₃ nanoparticles.

FIG. 13b shows fluorescence emission spectra for pure Eu:Y₂₀₃ nanoparticles excited at 260 nm showing fluorescence lifetime on order of 2 msec.

FIG. 14 illustrates magnetic characteristics of Co:Fe₂O₃ and Co:Nd—Fe₂O₃ powders synthesized by spray pyrolysis with different partial Co, Nd.

FIG. 15 is a schematic description of one embodiment of the synthesis of core/shell particles.

FIG. 16 shows a bright field TEM image of Co:Nd:Fe₂O₃/Eu:Gd₂O₃ core/shell particles.

FIG. 17a illustrates a comparison of magnetic characteristics of Co:Nd:Fe₂O₃ powder with Co:Nd:Fe₂O₃/Eu:Gd₂O₃ core/shell particles and Eu:Gd₂O₃ particles.

FIG. 17b illustrates an emission spectrum of Co:Nd:Fe₂O₃/Eu:Gd₂O₃ core/shell particles under excitation at 260 nm.

FIG. 18a shows an X-ray diffraction (XRD) spectrum of the primary Nd:Co:Fe₂O₃ particles compared to the typical XRD peaks of Fe₃O₄.

FIG. 18b shows an XRD of the core/shell Nd:Co:Fe₂O₃/Eu:Gd₂O₃ particles

FIG. 18c shows typical XRD spectral peaks of Fe₂O₃.

FIG. 18d shows the typical XRD spectral peaks of monoclinic Gd₂O₃.

FIG. 19 shows emission spectra of Co:Nd:Fe₂O₃/Eu:Gd₂O₃ core/shell particles and the IgG-Alexa Fluor 350 bound to their surface (excitation at 350 nm).

FIG. 20 illustrates saturation of a capture antibody (anti-rabbit IgG) immobilized on the surface of magnetic luminescent nanoparticles with rabbit IgG-Alexa Fluor 350. Absolute measured intensity of the Alexa peak (_) is compared to the intensity ratio Alexa/EuGd2O3 (.). The ratiometric approach reduces the uncertainty that arises from variations in the amount of particle separation from the sample with the magnet.

FIG. 21 shows a calibration curve for a competitive magnetic immunoassay for rabbit IgG. The signal of the labeled antigen (rabbit IgG-Alexa Fluor 350) bound on the surface of the magnetic nanoparticles is normalized by the Eu luminescence of the particles.

FIG. 22a illustrates an excitation spectrum of IgG-488 (primary) excited at 480 nm.

FIG. 22b illustrates an excitation spectrum of IgG-635 (secondary) excited at 620 nm.

FIG. 22c illustrates an excitation spectrum of the labels of FIGS. 22a and 22b, excited at 260 nm, using different analyte concentrations (0.2-25.6 μg/ml).

FIG. 23 illustrates a standard concentration base curve showing the variation of fluorescence intensity ratio with increasing analyte concentration.

FIG. 24 illustrates a standard time base curve for immuno-reaction saturation limit.

FIG. 25 illustrates results for immunoassays run inside a microchannel with and without (control) using electromagnets for mixing) with different target antigen concentrations (a) 0.1 μg/ml (b) 0.2 μg/ml, (c) 0.4 μg/ml, (d) and (e) 1.6 μg/ml.

The figures depict various embodiments of the present invention for purposes of illustration only. One skilled in the art will readily recognize from the following description that alternative embodiments of the structures and methods illustrated herein may be employed without departing from the principles of the invention described herein.

### DETAILED DESCRIPTION OF THE PREFERRED EMBODIMENTS

### Advantages and Utility

Briefly, and as described in more detail below, described herein are methods, and apparatus for performing an assay with internal luminescent calibration.

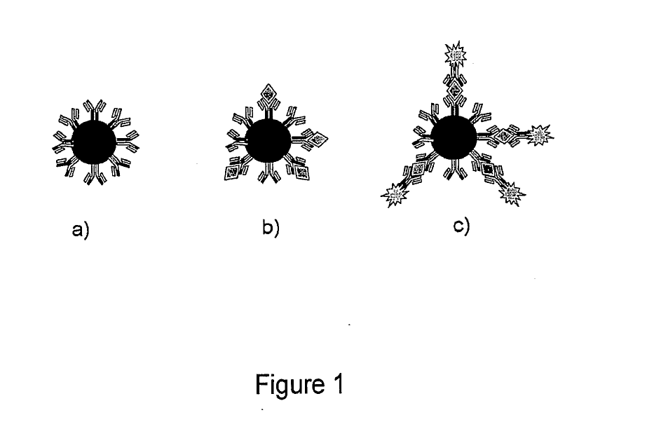

Referring to FIG. 1, according to one aspect of the present invention a first labeled composition which specifically binds with the analyte of interest (e.g. toxin) comprise a label and a composite particle with a magnetic core and luminescent shell (FIG. 1a). In the example shown, the first labeled composition comprises primary antibodies immobilized on the surface of the composite particle. The particles are incubated with a sample solution containing an unknown concentration of an analyte. During incubation, the analyte molecules, if any are present in the sample, bind to the composite particles, or antibodies (FIG. 1b). Next, the particles are incubated with a second labeled composition, in this example a secondary antibody labeled with a secondary fluorophore, the secondary antibody also specific to the analyte (FIG. 1c). The intensity of fluorescence is measured for the secondary fluorophore (12) and of the magnetic/luminescent particles (I₁). The ratio (I₂/I₁) is proportional to the concentration of the analyte and is normalized to the number of the measured particles, respective to the amount of primary antibodies. This aspect of the present invention helps to eliminate experimental error due to potential particle loss. While FIG. 1 illustrates a sandwich immunoassay involving a capture antibody and a second, labeled antibody, both of which bind to the analyte of interest, one of ordinary skill will readily appreciate that the present invention encompasses any type of binding assay in which an analyte of interest can be specifically bound to a ligand on the surface of the particles of the present invention. This includes assays in which the ligand is not an antibody, but can be a non-antibody receptor molecule, such as, e.g., biotin, avidin, a polynucleotide, a polysaccharide, a lipid, etc. and also includes assays in which the analyte itself is directly labeled with any type of detectable label, including, e.g., an enzymatic label, a fluorescent dye label, a fluorescent protein label (e.g., a fusion protein comprising green fluorescent protein (GFP), or the like. According to the present invention, a label is “associated with” an analyte if the analyte is directly labeled or indirectly labeled through, e.g., a second, labeled antibody. Each of these assays is well within the level of an ordinarily skilled artisan having the benefit of the present disclosure.

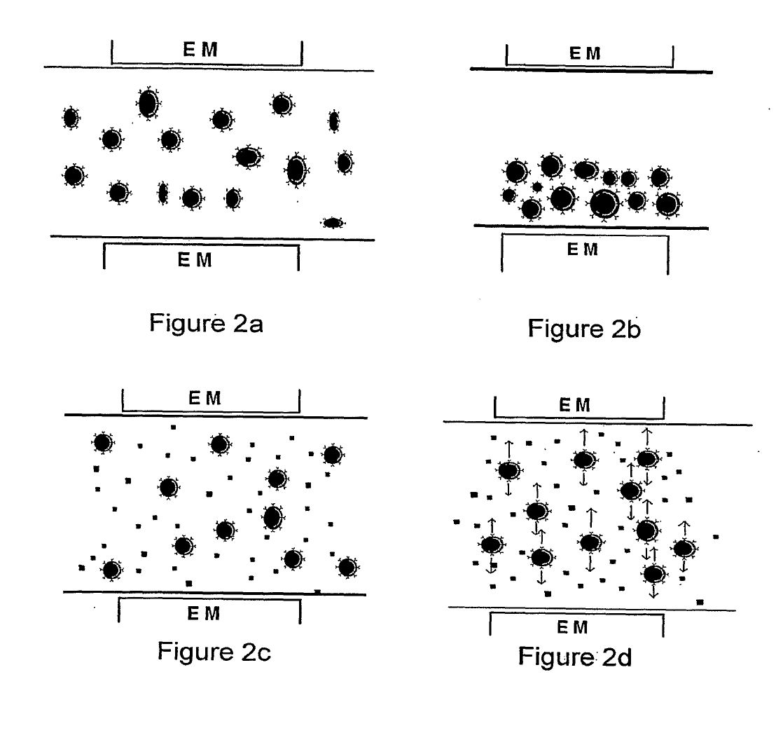

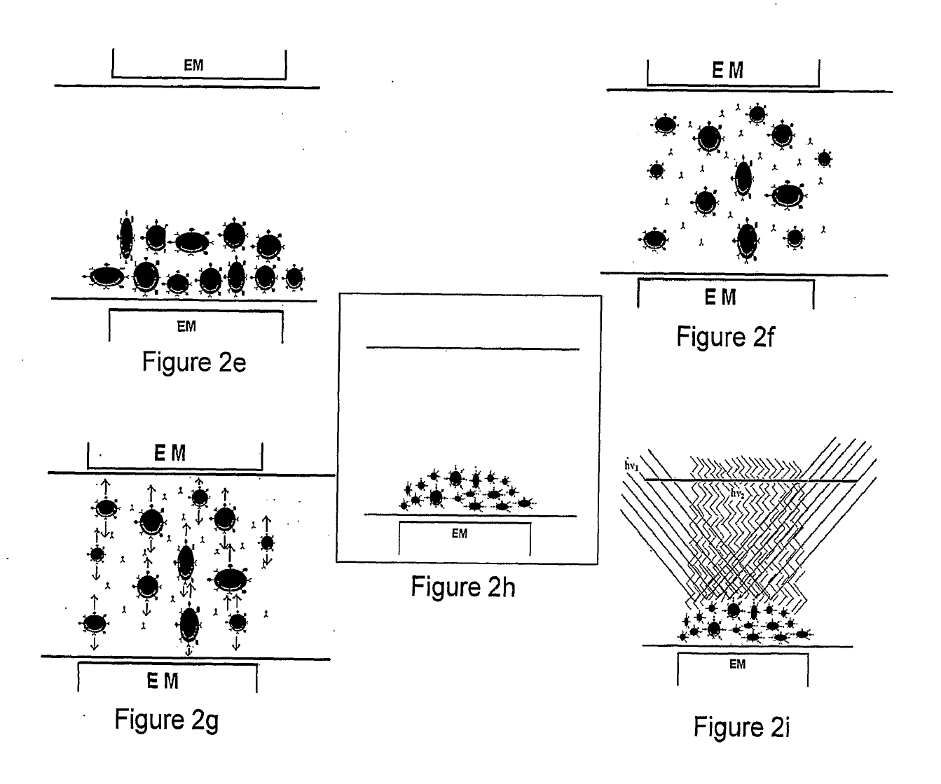

Referring now to FIG. 2, according to another aspect of the present invention, an assay is performed in a microchannel with external electromagnets for accelerated mixing. A microchannel, as used herein, is a small cavity, preferably having a cross section measurement ranging from about 50-100 microns. Operationally, the microchannel dimensions are dictated by the functional requirements that the microchannel be sufficiently wide as to accommodate the free movement of nanoparticles, and sufficiently narrow so that a magnetic field sufficiently strong to immobilize the nanoparticles can be localized within the microchannel. The microchannel can assume any shape consistent with meeting these requirements, including orthogonal, circular, spherical, etc. The microchannel can be formed by various means, e.g., the walls may be formed of transparent glass as described in the Examples, or by small capillary tubing. According to one embodiment, the first labeled composition (e.g., particles and primary antibody) is introduced into the microchannel (FIG. 2a) and is attracted to an energized electromagnet (FIG. 2b). Next the analyte is introduced into the channel (FIG. 2c) and the two magnets are alternatively switched on and off, thus inducing movement of the particles up and down towards whichever magnet which is in the on-state (FIG. 2d). This movement enhances the interaction between the first labeled composition and the analyte and therefore accelerates the immunoreaction. For the next step, one of the magnets is switched on while the other is off, thus holding the particles during the washing (FIG. 2e) and introduction of the second-labeled composition (e.g., secondary antibody and label) into the channel (FIG. 2f). During the incubation, the magnets are again used for inducing vibrating movements of the particles as described above (FIG. 2g). Finally, after washing (FIG. 2h), according to one embodiment, the two magnets are switched off and the particles are dragged by the flow down the channel where they are held by a third magnet for detection (FIG. 2i). In this example, the detection takes place through the wall of the channel, which is made from transparent glass, using a laser to excite the fluorophores. These aspects of the present invention all the whole assay to be performed in very small volumes, using very small amounts of the respective reagents. As a result, the speed of the assay is significantly increased, with incubation times in the range of several minutes. The internal luminescent calibration eliminates experimental error resulting from particle loss.

An important advantage of the proposed assay on the particle surface is that it permits any type of fluorophore to be used as an analyte label. The intensity of the secondary label can be tuned by varying the amount of used core/shell particles, and therefore the amount of surface binding sites. This way, more particles (larger surface) can be used in order to obtain higher intensity of the secondary label which will not change the quantitative (ratiometric) measurement but will increase the sensitivity. In addition, by using a large number of particles in the assay error from particle size non-uniformity is minimized because the size distribution of the particles does not change and hence the total particle surface area per unit mass of particles is constant. The sharp emission spectrum and long lifetime of Eu³⁺ ions (about 1 ms) allows us to use any other fluorophores as secondary labels such as quantum dots or polystyrene beads. Even if their spectra overlap with that of the Eu³⁺ ion, the signal from the long-lifetime Eu³⁺ ions can be resolved with time-gated detection.

The advantages provided by the synthesis method described herein useful in conjunction with the methods of the present invention include: a simple and low-cost single-step process is used to produce nanoparticles that are more uniform and less prone to aggregation than those produced using prior art methods such as ball milling or solution phase syntheses; the functionalization methods disclosed also are simple and low-cost and result in high quality nanoparticles and largely avoid agglomeration problems such as encountered with similar procedures that take place in the liquid phase, and greatly reduces or eliminates the need for post-functionalization washing of the nanoparticles; and because the spectral properties of the nanoparticles of the present invention do not depend on the particle diameter, the size distribution of a population of the particles need not be monodisperse. Nanoparticles, as used herein in conjunction with the present invention, are particles that are less than 1 micron in diameter, preferably less than 500 nm, and more preferably 100-200 nm.

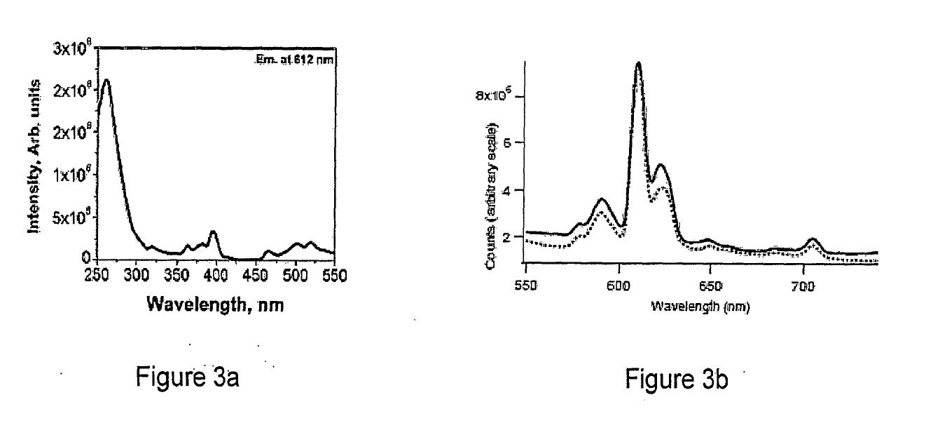

According to some embodiments, Europium oxide (Eu₂O₃) particles are used. These particles have a useful excitation region from about 250-410 nm with a maximum at 260 nm as seen in FIG. 3a. Other excitation peaks are located near 395 and 466 nm. After excitation, the Eu₂O₃ particles produce an emission peak centered at 612 nm. The emission spectrum has the following salient characteristics, typical of europium and its chelates: (1) large Stokes shift (144 nm or 216 nm, depending on excitation wavelength); (2) a narrow, symmetric emission feature at 612 nm (full width half maximum, FWHM, of 8 nm); (3) a long lifetime (in this case measured with a time resolved fluorescence system to be about 300 μs). Excitation and emission spectra are shown in FIGS. 3a and 3b. Longer emission lifetimes (up to about 1 ms) can be achieved by doping Eu atoms into appropriate host materials, e.g. Y₂O₃ or Gd₂O₃.

Nanoparticles of simple lanthanide oxides can offer all the advantages of lanthanide chelates without the complex synthesis and the somewhat uncertain composition, the latter issue leading to uncertainties in the conjugation chemistry. Due to their chemical inertness, and the fact that the lanthanide ion is sequestered in a crystal lattice, they are not susceptible to photo-bleaching or oxygen quenching. The synthesis method disclosed herein allows production of a range of lanthanide oxides that span the useful optical spectrum. The lanthanide family offers multi-wavelength labels for multiplexed assays with high throughput, using a number of the lanthanides (Eu-red, Tb-green and Dy-blue, Sm-red) doped into suitable host materials such as Y₂O₃.

The advantage offered by simple, single material nanoparticles can be extended by multi-functional materials that are engineered on the nanoscale. The multiple properties may include optical, magnetic, and electrical characteristics that can be usefully employed in an assay. A magnetic moment can allow a particle to be separated from its matrix prior to measurement.

The use of magnetic beads is not new, nor is the use of lanthanide labels for immunoassays in food monitoring. The implementation of magnetic bead separation with fluoroimmunoassays for pathogen detection is being pursued, for example, at the USDA Eastern Region Research Center. Tu et al. [55] recently reported a clever scheme to use Eu and Sm (samarium, another lanthanide) for the detection of E. coli 0157 and Salmonella on alfalfa seeds with an immunoassay. Magnetic beads were used in a sandwich assay in which the beads were separated with a magnetic concentrator. Antibodies that were labeled with Eu and Sm were incubated to bind with the E. coli and Salmonella. A chelation step was then necessary to remove the lanthanide ions from the antibodies prior to detection. The authors made use of the long phosphorescence lifetime of the lanthanide chelate to carry out gated detection of the emission. Sensitivity and specificity were good but the procedure was complicated and impeded by the requirement for multiple steps, especially the chelation step.

The present invention combines magnetic and fluorescence properties in a nanoparticle label. One approach is to use polymer beads with a magnetic core and doped with a fluorophore. Mulvaney [56]; Wang [57]. Drawbacks to this approach are that the beads are large (0.8 μm), subject to photobleaching with organic dye-doped beads, and are expensive. Our proposed method of production combines magnetic and fluorescent properties into one nanoparticle in a controlled manner. They can provide low cost reagents with the ability to perform multiplexed assays.

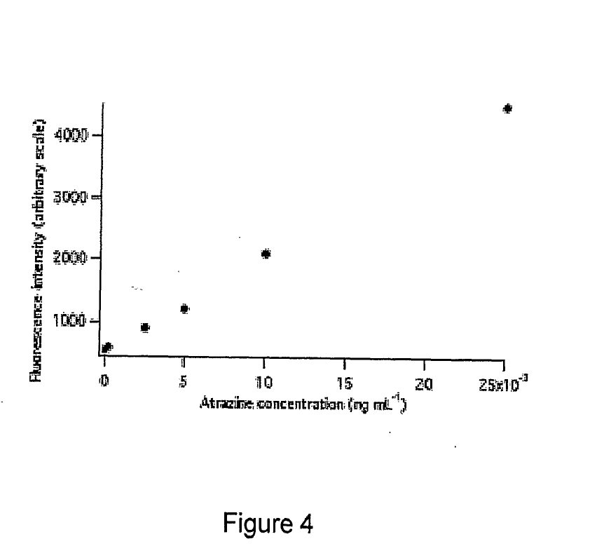

Few others have succeeded in applying fluorescent nanoparticle formats in a quantitative manner. Feng [49]. Generally, quantum dots and similar materials have been used primarily for qualitative labeling and staining in microscopy. Qhobosheane [58]. However, the use of simple Eu₂O₃ nanoparticles in a fluoroimmunoassay for atrazine is described herein. Feng [49]. The results are shown in FIG. 4. The limit of detection in a non-optimized assay was superior to a standard ELISA, but without the tedious preparation and processing. The methods of the present invention provide considerable improvements in, e.g., assays for toxins such as botulinum toxin, ricin, abrin, or to detect infective prions in food.

Small equipment size permits parallel processing of samples with a range of bioassays that are designed to detect a variety of food toxins and pathogens. Miniaturized systems permit faster analyses than are otherwise possible, with less time-consuming human involvement in the various steps. For example, the rate of diffusion of analytes to surfaces scales with the dimensions of the system to the second power. Hence, diffusion times, that may limit rates of binding of Ag (antigen) to Ab (antibody) in some cases, are reduced by a factor of 100 if the length scale for diffusion is reduced by a factor of 10.

While miniaturization offers considerable benefits in terms of size, portability, cost, automation and throughput, further advances in detection technology are afforded by recent developments in nanotechnology. The engineering of materials on the nanoscale provides new properties and functionalities that were not possible in the past. The combination of nanotechnology with microfabricated miniaturized analysis systems opens up new vistas for securing our nation's food supply.

Thus, the inventive materials and methods described herein system provide the following advantages: (1) the working volumes and the amounts of consumed reagents are reduced by several orders of magnitude; (2) the time for magnetic separation is significantly reduced because the solution will be situated very close (a few micrometers) to the magnet surface, where the magnetic field is strongest and the magnetic particles will only need to move a very short distance in order to be separated from the solution; (3) multiple parallel microchannels may be fabricated on a single substrate and controlled simultaneously by one electromagnet. This allows many parallel assays to be performed at the same time; (4) poly(dimethylsiloxane) (PDMS)-based channels can be made cheaply enough to be disposable; (5) the use of an electromagnet to switch electrically between retention (separation) and elution regimes of the nanoparticles in a continuous flow eliminates the need for stop-flow and waiting periods and decreases the reaction time since diffusion no longer is a rate limiting step for binding; (6) the excitation/detection of the fluorescent signal can be performed through the channel wall as the particles are attracted by the magnet. Excitation by an evanescent wave on the top of a waveguide structure can be used to restrict excitation to only the nanoparticles that are closest to the wall. In another embodiment “off-chip” detection in a plate reader after the elution of particles can be used to provide fully automated devices for precise and high-throughput detection.

### Introduction

Nanoparticles have found tremendous applications in the field of biotechnology. Their unique size-dependent properties make these materials superior and indispensable in many areas of human activity. Out of many size-dependent properties available optical and magnetic effects are the most used for biological applications. [59, 60] Due to their controllable size ranging from a few nanometers up to tens of nanometers, comparable to most of the cell parts and even close to the size of proteins, nanoparticles can be used to probe the cell machinery without introducing too much interference. Applications of nanomaterials to biology or medicine includes fluorescent biological labels [61-63], Drug and gene delivery [64], Bio detection of pathogens [65], Detection of proteins [66], Probing of DNA structure [67], Tissue Engineering [68], Tissue destruction via heating [69], Separation and purification of biological molecules and cells [70], MRI contrast enhancement [71] and phagokinetic studies.

A nanoparticle in itself is rarely sufficient for the above-outlined uses. Usually, a nanoparticle must be coated with a layer of material that functions as a bioinorganic interface. The coating may include antibodies, biopolymers like collagen or monolayers of small molecules which make the nanoparticles biocompatible. In addition the particles should be either fluoresce or magnetic for detection purposes. Nanoparticles can also be fluorescent-dye coated for optical detection. A nanoparticle usually forms the core of nano-biomaterial. It can be used as a convenient surface for molecular assembly and may be composed of inorganic or polymeric materials. The core itself might have several layers and be multifunctional. For example, combining magnetic and luminescent layers one can both detect and manipulate the particles.

The core particle is often protected by several monolayers of inert material. Organic materials that are adsorbed or chemisorbed on the surface of the particle can serve the same purpose and also act as a biocompatible material. However an additional layer of linker molecules is required for further functionalization. This linker molecule has reactive groups at both ends. One group is aimed at attaching the linker to the particle surface and other is used to bind various molecules like biocompatibles, antibodies, fluorophores etc. depending on the function required by the application.

Magnetic nanoparticles offer some attractive possibilities in the field of biosciences as they can be manipulated by an external magnetic field gradient. [72] External manipulation and ability of magnetic fields to penetrate most of the bio-surfaces leads to many applications involving the transport and/or immobilization of magnetic nanoparticles or magnetically tagged nanoparticles. They can be used to deliver drugs to a targeted region [73]. The magnetic particles can be made to resonantly respond to a time varying magnetic field consequently transferring energy from the exciting field to the particle itself, so they can be used as hypothermia agents for targeted bodies such as tumors [69]. Also because of there easy maneuverability, small size and direct detection, magnetic nanoparticles have found use as labels in new generation of immunoassays [74]. Magnetically labeled targets are detected directly with a magnetometer [75, 76], with a microscope based on a high transition temperature dc superconducting quantum interference device (SQUID) for rapid detection of superparamagnetic nanoparticles [77, 78]. In this technique a suspension of magnetic particles carrying antibodies is added to targets and the mixture is placed on the microscope. Magnetic field pulses are applied parallel to the SQUID which causes the nanoparticles to develop a net magnetization. This magnetization relaxes when the field is turned off. Unbound nanoparticles relax rapidly by Brownian rotation and contribute no measurable signal. Nanoparticles bound to the target are captured and undergo Nëel relaxation, producing a slowly decaying magnetic flux, which is detected by SQUID.

Recent technology research in the field immunoassays using magnetic-nanoparticles is toward accelerating the process [79], miniaturization of the system and alternative detection capability using magnetic microbeads loaded with fluorophores and quantum dots [80]. The present invention provides a novel assay inside a micro channel using magnetic nanoparticles coated with fluorophores as an assay surface and analyte detection using fluorescent-labeled antibodies. In most conventional immunoassays, observation and final quantification is based on absolute value of a signal that is proportional to the amount of captured analyte. Consequently, the number of incubation and washing steps used for the immunoassay can affect the final values, as a percentage of both analyte and detecting body will be lost during these procedures. Luminescent magnetic particles of the present invention (including fluorescent nanoparticles and particles conjugated to fluorescent dye) and coated with, e.g., an antibody provide an assay surface for, e.g., a sandwich format assay. The signal associated with the particle introduces an internal standard that can be used to improve assay result accuracy by normalizing the signals derived from the particle and the analyte for magnetic nanoparticles of known size range and with controlled coating procedures the number of antibodies attached on the surface can be characterized and used as a base for comparison.

In general, the nanoparticle compositions used in conjunction with the present invention comprise a metal oxide particle having a desirable optical property that has been coated with a functionalizing reagent. The functionalizing reagent used may comprise a silane as disclosed in co-owned pending U.S. Patent Publication 2003/0180780, incorporated herein by reference for all purposes, or comprise a protein or peptide such as, e.g., BSA or an immunoglobulin, or may be a polyionic polymer, such as, e.g., (poly-L-lysine hydrobromide, PL).

Preferred particle diameters are in the range of between about 10 and 1000 nm, more preferably between about 10 and 200 nm and even more preferably between about 10 and 100 nm, or between about 20 and 50 nm. In preferred embodiments, the metal oxide particles have the generic formula MeₓO_{y}, wherein 1≦x≦2, and 1≦y≦3, and wherein preferably, Me is a rare earth element selected from the lanthanide series and includes, but is not limited to, europium (Eu), cerium (Ce), neodymium (Nd), samarium (Sm), terbium (Th), dysprosium (Dy), gadolinium (Gd), holmium (Ho), thulium (Tm), or Me may be chromium (Cr), yttrium (Y), iron (Fe). Other suitable metal oxide particles include silicon oxide (SiO₂), and aluminum oxide (Al₂O₃) mixed with Eu₂O₃. or Eu³⁺.

In other preferred embodiments, the metal oxide particle comprises a doped metal oxide particle by which is meant a metal oxide, and a dopant comprised of one or more rare earth elements. Suitable metal oxides include, but are not limited to, yttrium oxide (Y₂O₃), zirconium oxide (ZrO₂), zinc oxide (ZnO), copper oxide (CuO or Cu₂O), gadolinium oxide (Gd₂O₃), praseodymium oxide (Pr₂O₃), lanthanum oxide (La₂O₃), and alloys thereof. The rare earth element comprises an element selected from the lanthanide series and includes, but is not limited to, europium (Eu), cerium (Ce), neodymium (Nd), samarium (Sm), terbium (Tb), gadolinium (Gd), holmium (Ho), thulium (Tm), an oxide thereof, and a combination thereof. Nanoparticles of such oxides may be manufactured according to the methods of the present invention, purchased from commercial suppliers, or fabricated using methods known to those of ordinary skill in the art.

The desirable optical properties of the compositions of the present invention include optical properties that allow the compositions to be useful as labeling agents, such as, e.g., fluorescence, fluorescence resonance energy transfer (“FRET”), and phosphorescence. Thus, the compositions of the present invention may be used by one of skill in the art in the same manner as fluorescent dyes, FRET pairs and other labeling reagents, but with the advantages that nanoparticles bring to labeling technology in terms of larger Stokes shift, longer emission half-life (for lanthanide-containing nanoparticles), diminished emission bandwidth, and less photobleaching as compared with, e.g., traditional fluorescent dyes.

Surface modification and other methods may be used in the practice of the invention for surface modification (i.e., functionalization) and conjugation of the nanoparticles of the invention. In one embodiment, surface modification and conjugation comprises direct coating of the nanoparticles with a protein such as, e.g., BSA, ovalbumin or immunoglobulin. In another embodiment, surface modification is accomplished by physical adsorption and functionalizing with a polyionic polymer such as, e.g., poly-L-lysine hydrobromide, PL.

Using appropriate buffer conditions (pH and concentration), a variety of proteins can be adsorbed spontaneously on the surface of the nanoparticles without affecting their fluorescence properties. The protein coated particles are purified by 3 rounds of centrifugation and are stable for more than 1 month in buffer solution. Adsorption of bovine serum albumin (BSA) provides multiple functional groups (amine, carboxylic) for covalent conjugation to other biomolecules using standard cross-linking procedures. If BSA-biotin is used as a coating protein, biotinylated particles are produced for a variety of applications in bioassays. If the particles are coated with BSA-hapten (small molecule), such as the coating antigens commonly used in ELISA, the modified particles may be used as fluorescent competitors in immunoassays. The nanoparticles are efficiently coated with immunoglobulin molecules, preserving the functionality of the nanoparticles and the functionality and activity of the immunoglobulins. The number of binding sites (biotin, hapten, antibody) may be controlled during the coating procedure by mixing a specific protein (i.e., the protein providing the binding site) and a non-specific blocking protein (i.e., one that does not provide a binding site) in different ratios. Blocking proteins are well-known to those in the biochemical arts and include, e.g., BSA, casein, milk proteins, and other agents useful for blocking non-specific binding in biochemical reactions such as, e.g., ligand binding assays, Western blots, ELISAs, etc. Examples of pairs of specific proteins and non-specific blocking proteins include, e.g. BSA-biotin:BSA, specific anti-rabbit IgG:non-specific sheep IgG. The blocking protein prevents possible non-specific binding of the nanoparticles to other proteins and/or surfaces during the performance of bioassays improving in this way the signal/noise ratio.

PL is a polycationic polymer that adsorbs spontaneously from aqueous solutions onto the negatively charged metal oxide surfaces via electrostatic interactions. The excess of PL is washed off by centrifugation. The formed layer of PL is stable under the most commonly used buffers. The introduced amino groups on the surface of the particles permit their conjugation to a variety of small molecules (haptens) and biomolecules with appropriate functionalizations.

The immunoassay technique is the fastest growing analytical technology for the detection and quantification of biomolecules. It takes advantage of affinity binding between antibodies and the corresponding antigens that allows the detection of one of these, even if it is present at very low concentrations and in complex biological matrixes such as whole blood, serum and other biological fluids. The measurement of binding reaction can be performed by monitoring changes in the different physical phenomenon associated with the biomolecules and the labels used, as well as the configuration of the assay.

The antibody-antigen specific reaction, which is the basis of all immunological techniques, can be characterized by its structure, its strength, also known as affinity, and its stability, also known as avidity. The detection and quantification of the antibody-antigen interaction can be achieved using a variety of different labels, present either in the antigen or antibody. Different techniques can be employed in the design and development of immunoassays. Radio labeled immunoassays use reagents incorporating radioisotopes as tracers to monitor the distribution of free and bound antigen in radioimmunoassay or free and bound antibody in immunoradiometric assays. Fluoroimmunoassays involve in situ detection with antibodies linked to the fluorescent label by using external laser excitation and then measuring the fluorescence signal or by taking optical micrographs. Enzyme immunoassays involve the use of enzyme activity as a means of detecting the binding of an antibody/enzyme conjugate. Enzymes are specific in both reaction they catalyze and the substrate they recognize and are subject to the regulation of their activity by other molecules. Enzyme immunoassays are similar to immunofluoroscence assays. Only difference is in type of detection, which is spectrophotometric when label produces colored product or electrochemical when enzyme catalyzes a redox reaction.

Several other techniques and configurations are used in immunoassay design such as chemiluminescence, which describes the emission of light that occurs as a result of certain chemical reactions producing high amount of energy which is lost in the form of photons when electronically excited product molecules relax to their stable ground state, light-scattering, based on the reaction between an antigen and an antibody to produce an aggregate large enough to scatter light to a greater degree than do the constituents of the reaction, electrochemistry, based on the measurement of the redox potential, by measuring either a current or potential of a reaction, disposable tests that are often membrane based assays which provide visual results and can be designed as rapid tests.

There are three basic configurations of an immuno-reaction, with either antibodies or antigens present in the sample to be analyzed. In the sandwich configurations of an immuno-reaction, the capture antibody is immobilized onto the surface and binds to the antigen present in the sample. A second antibody labeled with a fluorophore binds to a different epitope of the antigen, leading to a fluorescent signal that is proportional to the amount of antigen present in the sample. In some embodiments of the present invention a sandwich configuration immunoassay is used. The use of different labels for the capture bead and secondary antibody provides the ability to normalize analyte signal to capture bead signal, thereby improving the accuracy of the assay result.

### Theoretical Model

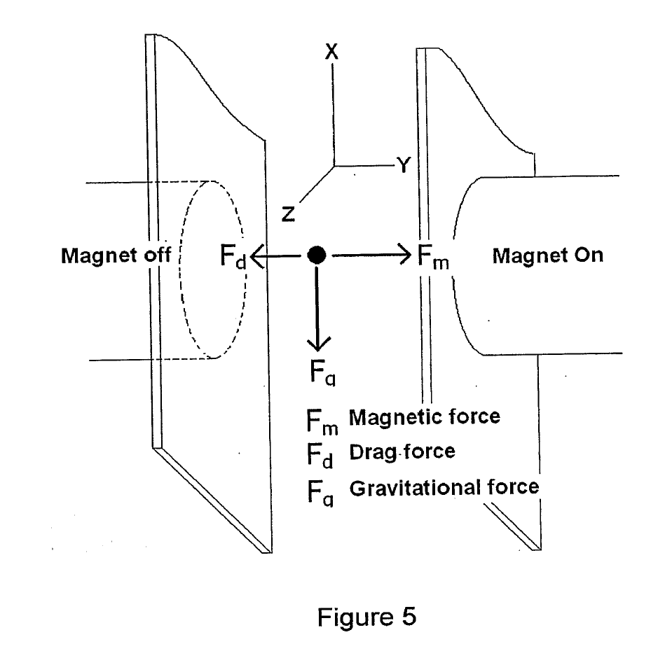

The movement of micron sized magnetic particles in a non-flowing aqueous solution can be classified as quasi-static motion (no inertial effects), because of high viscous forces acting on them. See FIG. 5. Therefore the particle moves under the influence of viscous drag, F_{d} and the magnetic force, Fₘ. The magnetic force on the particle is given by [8])

Fₘ=(VΔχ/2μₒ)grad(B²) (1)

where Δχ is the volumetric susceptibility difference between particle and solution, V is the particle volume and μₒ is the magnetic permeability of free space. Also from vector calculus for static irrotational field grad (B²)=2B grad (B). The drag force on the particle in a fluid is given by Stokes equation

F_{d}=3πηDₚυ_{y} (2)

where η is the fluid velocity, Dₚ is the particle diameter and υ_{y} is the component of particle velocity in the direction of magnetic field application. The force balance on a particle yields

(mass)*(acceleration)=Fₘ−F_{d}

Using equations (1), (2) & (3) we get the following differential equation in terms of particle position

d²y/dt²+[(π²dₚ⁴η)/(2ρ)]dy/dt−[(Δχπ²Dₚ⁶)/(36μₒρ)]Bgrad(B) (4)

### Materials and Methods of the Invention

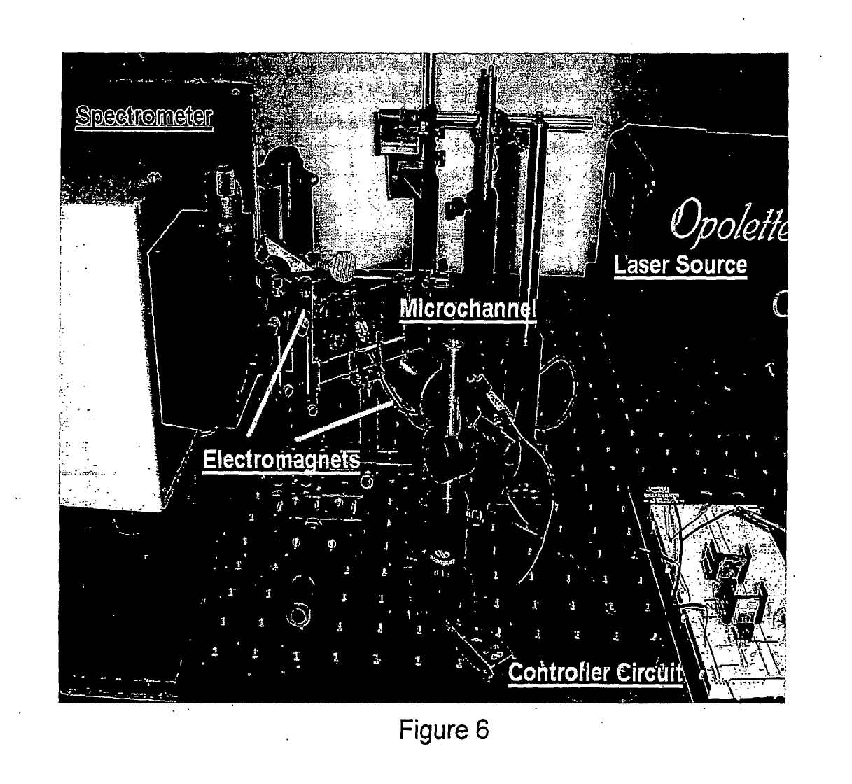

Referring to FIG. 6, an experimental apparatus for forming a microchannel useful in conjunction with the present invention is shown. The major components the apparatus include a microchannel set up, electromagnets, a controller circuit, a laser source, and a spectrometer. Two 12 VDC round electromagnets (part number ER1-103) from Dura Magnetics, Inc. were used across the channel. The supply voltage for these magnets can be varied from 12-24 V which corresponds to a current variation of 0.2-0.5 amps. For the experiment the magnets were supplied with 18 V which corresponds to 0.334 amp current thru the coil. The magnetic flux intensity at the edge of magnets was found to be 80 mTesla. Magnetic flux was concentrated at the central part of the channel where the magnetic particles were held. At the point of application the two magnets were separated by a distance equaling the sum of channel depth and twice the thickness of glass. The electromagnets were screwed to a holder with the capability of moving in and away from the channel. The channel was held in a vertical position such that the gravitational force and magnetic force were acting perpendicular to each other.

Two NPN transistors (NTE263), a NAND logic gate (74HCT04N, Philips) were used in circuitry to alternatively turn on the two magnets. Signal for switching was generated using 15 MHz arbitrary waveform generator (Agilent 33120A) which was also used to specify the frequency at which the electromagnets switch. Laboratory DC power supply (GPS-3030D, GW) was used to power the NAND logic gate and the electromagnets.

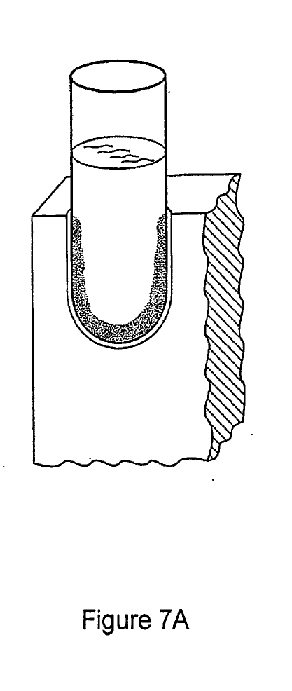

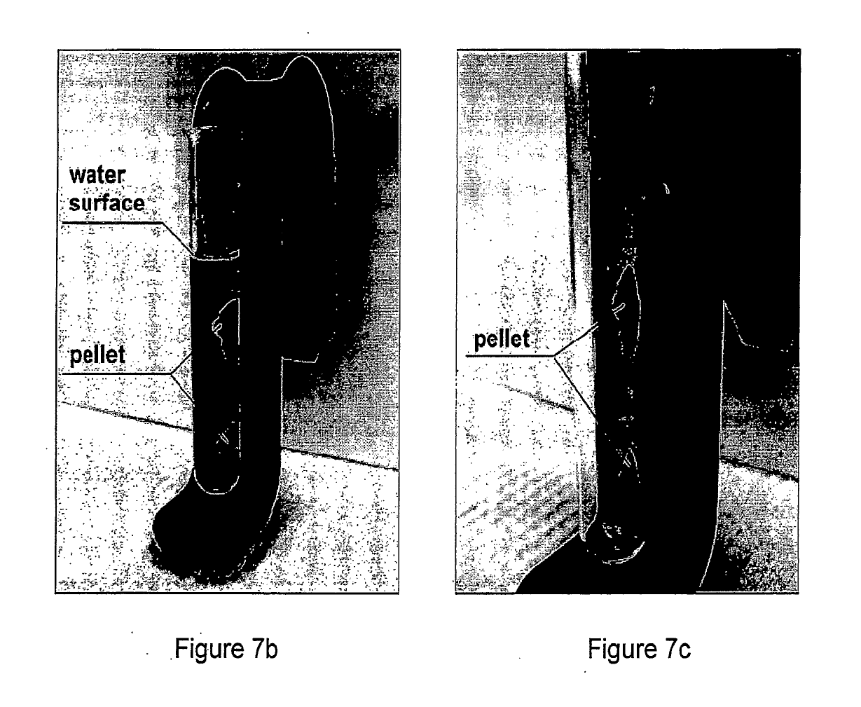

Ohmicron magnetic rack (Strategic Diagnostics, Newark, Del.), such as shown in FIGS. 6, 7, was used for magnetic particle separation. Fluorescence measurements for conventional immuno-assay were performed using Spectramax M2 cuvette/microplate spectrofluorometer (Molecular Devices, Sunnyvale, Calif.) in black 96-well plates from Nuncand the spectrums were analyzed using SoftMax pro 4.7 (Molecular Devices) and WinSpec/32 (Microsoft corps.).

A high aspect ratio (ratio of channel length to channel depth) microchannel was used to get the closest possible approximation of a perfect laminar flow. The channel was fabricated using two glass slides (75×50×0.9 mm, Corning Glass Works) with epoxy as spacer, which also defined the walls of the channel. Inlet and outlets ports were drilled in the glass for external fluid connections. The channel was rectangular in shape with dimensions 60 mm/10 mm/110 μm (length/width/depth) with approx. volume of 40 μl.

The magnetic particles used in one example comprise 2.52% solids-latex (by wt) of polystyrene Superparamagnetic microspheres, (1-2 μm, Polysciences). The antibodies used were Alexa Fluor-488 anti-mouse IgG, whole molecule developed in goat (Molecular Probes) and Alexa Fluor-635 anti-mouse IgG, whole molecule developed in goat (Molecular Probes), 1×PBS (0.1M PBS diluted 1:10 with deionized water (18 mΩcm), PBS: 90 g/L NaCl, 10.9 g/L Na₂HPO₄, 3.2 g/L NaH₂PO₄ in deionized water), Borate buffer (12.4 g/L Boric acid, 19.1 g/l Sodium Tetraborate in distilled water, pH adjusted to 8.5 using NaOH). The antigen used was Mouse IgG (SigmaAldrich, St Louis, Mo.).

A channel is formed by a glass substrate and a poly(dimethylsiloxane) (PDMS) layer bonded to the top of the glass The PDMS—based microfluidic devices is fabricated by placing a PDMS template in contact with the glass surface and applying pressure to create a fluid-tight and air-tight seal in the manner of Chabinyc et al. [82]. The reagents for the assay are introduced into the microchannel with a flow rate controlled by automatic syringe pump.

Functionalized magnetic nanoparticles interacted with the sample within a reservoir built on the same substrate as a part of the microfluidic system. Following the binding reactions in the reservoir, the magnetic nanoparticle substrates were separated from the rest of the sample by magnetic retention at the channel wall. Flow rates were optimized in such a way that long enough time will be given for binding reactions to occur in the reservoir and magnetic particles to be separated in the channel. This can be achieved by varying the ratios between the volumes of the reservoir and the channel. For given flow rate, increasing the reservoir volume will give longer times for the binding to occur.

### Magnetic Assay

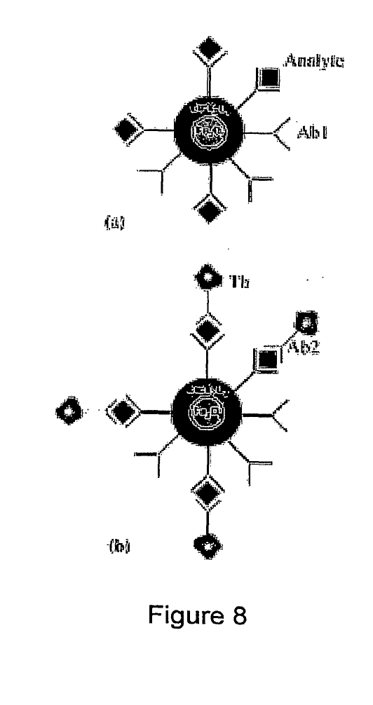

Magnetic separation assays use magnetic beads to facilitate the separation of bound labeled molecules from the free molecules in the solution. Taking advantage of the unique combination of magnetic and optical properties of our nanoparticles, a sandwich immunoassay for toxins (proteins) was performed on the surface of magnetic Eu nanoparticles coated with a capture antibody, as shown in FIG. 8. The enormous surface area presented by mobile nanoparticles in suspension as a substrate was used for immobilization of the biorecognition elements. The nanoparticles were dispersed in the sample solution and used as probes to capture any analyte that is present in a sample. A secondary antibody or antibodies labeled with another fluorophore (secondary label), with an emission wavelength that is different from that of Eu was used to detect the bound analyte. The secondary label can be an organic fluorophore (i.e., Alexa Fluors or cyanine dyes), other lanthanide nanoparticles (Dy:Y₂O₃ or Tb:Y₂O₃), or a quantum dot. After the secondary antibody bound to the analyte on the magnetic particle surface, the magnetic separation was performed. The fluorescence spectrum of the extracted immunocomplex shows different intensities of the peaks corresponding to Eu magnetic particles and to the secondary antibody labels as described herein. The peaks of the secondary labels are compared to that of Eu magnetic particles, which serve as an internal standard; the Eu signal indicates the amount of capture antibody available in the mixture. The signal from the second fluorophores indicates the amount of analyte captured. The assay may be carried out in a microwell format or in a microfluidic device.

### Optimization of a Magnetic Sandwich Fluoroimmunoassay

In one aspect of the present invention, the fluoroimmunoassay based on magnetic Fe₂O₃/Eu:Y₂O₃ nanoparticles was tested in a sandwich format in buffer solution for the detection of model proteins (IgG, BSA or botulinum toxoid) using secondary antibodies labeled initially with organic dyes (e.g. Alexa Fluor or cyanine dyes). To achieve a highly sensitive assay, the parameters of the immunoassay performance were optimized for the magnetic separation and the fluorescence detection of the internal standard (the Eu magnetic nanoparticle) and the second fluorescent label. The conditions for efficient magnetic extraction, such as the amount of magnetic nanoparticles used and time of separation, were determined. In addition, the amount of antibody immobilized on the surface of the magnetic nanoparticles, the optimal concentration of the secondary antibody, and the kinetics of the immunoassay were crucial in determining the dynamic range and the detectability of the assay. The detection parameters were optimized to achieve high signal/noise ratio using the least concentration of immunoreagents. The analytical performance of the magnetic based immunoassay, such as precision, accuracy and reproducibility, was be evaluated with these conventional fluorophores labels.

Several detection schemes were evaluated. The organic dyes have short lifetimes compared to the magnetic Eu nanoparticle substrate. Using time-gated detection we make use of the difference in lifetime of emission from the highly efficient organic dye labels compared to the long lifetime lanthanide to detect the potentially weaker emission from the Eu substrate. In some embodiments, lanthanides or quantum dots are used as secondary labels. The long lifetimes of lanthanides allow time-gating of detection and thereby the ability to discriminate against strong background fluorescence that may be encountered in measurements in foods. Among the lanthanides terbium (Th-green), dysprosium (Dy-blue) and samarium (Sm-red) nanoparticles were explored as secondary antibody labels. In some aspects, quantum dots are used to probe up to six or eight different analytes on a single Eu substrate particle.

Depending on the fluorophores used, a large difference may be present between the intensities of the detected fluorophores. The intensity of the secondary label should preferably be comparable to the intensity of the Fe₂O₃/Eu:Y₂O₃ nanoparticles. Possible differences in intensities can be overcome by controlling the binding sites per Fe₂O₃/Eu:Y₂O₃ particle and by optimizing the total number of particles used. If the intensity of the secondary label is too low compared to Fe₂O₃/Eu:Y₂O₃ nanoparticles, the number of binding sites per particle are increased and the amount of particles used are reduced, or vice versa. Finally, the difference in lifetimes between fluorophores or quantum dot and the lanthanide substrate nanoparticle can be used to optimize the sensitivity of the assay.

Reducing non-specific binding and minimizing nanoparticle agglutination are also crucial steps in using nanomaterials as labels in sensitive and reproducible bioanalysis. There is no universal solution to this difficult problem. The critical parameters are the charge, size and surface functionality of the nanoparticles and the buffer (sample). The surfaces of the nanoparticles were efficiently blocked with proteins to avoid non-specific binding in immunoassays, as has been demonstrated with earlier microprinting experiments. Dosev [50].

The demonstration of nanotechnology with conventional laboratory instrumentation such as micro plate readers was the first step towards the development of a useful, portable, and automated measurement system. Miniaturization was the next logical step. Once the proposed assays based on magnetic fluorescent nanoparticles were optimized, they were downscaled to nanoliter sample volumes. Magnetic bead immunoassays have been performed in volumes of several μL using a capillary tube [31]. Therein, Zhao used commercially available magnetic beads that were not fluorescent and measured the fluorescent signal from the supernatant. This indirect detection approach suffers from lack of precision and poor reproducibility.

Table 1 provides a non-limiting listing of the reagents, abbreviations for the reagents, formulae, suppliers, form of usage of the reagent in the described syntheses and examples of alternative reagents useful for producing the particles and for practicing the methods of the invention. The listing is intended to be exemplary and to provide guidance to an ordinarily skilled artisan as to other materials useful for practice of the invention. Those materials are readily ascertained by the ordinarily skilled artisan provided with the teachings of this specification.

TABLE 1
Exemplary Reagents

| | | | Form of | |
|---|---|---|---|---|
| | | | Usage in | Substitute |
| Reagent | Formula | Supplier | Synthesis | Reagent |
| Tris(2,2,6,6- | | Alfa | Vapor at | Europium metal, |
| tetramethyl-3,5- | | Aesar, | 200 C. | any europium |
| heptanedionato) | | Ward | | compound that |
| europium(III) | | Hill, | | has sufficient |
| abbreviated | | MA | | vapor pressure at |
| as Eu(TMHD)₃ | | | | 200 C. and does |
| | | | | not decompose |
| | | | | below 400 C. |
| Sodium metal | Na | | Vapor at | Other alkali or |
| | | | 400 C. | alkaline earth |
| | | | | metals |
| Zinc metal | Zn | | Vapor at | Other alkali or |
| | | | 400 C. | alkaline earth |
| | | | | metals |
| Europium | Eu(NO₃)₃•6H₂O | Alfa | Aqueous | Other soluble |
| (III)nitrate | | Aesar, | solution or | europium salts, |
| | | Ward | solution in | such as EuCl₃, |
| | | Hill, | an organic | that does not |
| | | MA | solvent that | negatively affect |
| | | | is readily | the synthesis |
| | | | nebulizable | reactions |
| Ytrium | Y(NO₃)₃•6H₂O | Alfa | Same as | Other soluble |
| (III)nitrate | | Aesar, | above | europium salts, |
| | | Ward | | such as YCl₃, |
| | | Hill, | | that does not |
| | | MA | | negatively affect |
| | | | | the synthesis |
| | | | | reactions |
| Terbium | Tb(NO₃)₃•6H₂O | Alfa | Same as | Other soluble |
| (III)nitrate | | Aesar, | above | europium salts, |
| | | Ward | | such as TbCl₃, |
| | | Hill, | | that does not |
| | | MA | | negatively affect |
| | | | | the synthesis |
| | | | | reactions |
| Hexamethyl- | C₆H₁₈OSi₂ | Sigma | Both as the | Any other |
| disiloxane | | Aldrich, | vapor and a | organic |
| abbreviated | | St. | solution in | compound that |
| as HMDS | | Louis, | an organic | contains silicon |
| | | MO | solvent, | and has |
| | | | such as | sufficient vapor |
| | | | ethanol, | pressure at room |
| | | | that is | temperature and |
| | | | readily | is soluble in the |
| | | | atomizable | solvent used for |
| | | | | dissolving the |
| | | | | other starting |
| | | | | materials that |
| | | | | does not |
| | | | | negatively affect |
| | | | | the synthesis |
| | | | | reactions |
| (3- | | Sigma | Vapor at | Many other |
| Aminopropyl)tri- | | Aldrich, | room | silanizing |
| ethoxysilane | | St. | temperarure | reagents. |
| abbreviated | | Louis, | | |
| as APTES | | MO | | |
| (3- | | Sigma | Vapor at | Many other |
| Aminopropyl)tri- | | Aldrich, | room | silanizing |
| methoxysilane | | St. | temperature | reagents. |
| abbreviated | | Louis, | | |
| APTMS | | MO | | |
| Iron(III) | Fe(NO₃)₃•9H₂O | Sigma | Aqueous | Other soluble |
| nitrate | | Aldrich, | solution or | iron salts such as |
| | | St. | solution in | FeCl₃. |
| | | Louis, | readily- | |
| | | MO | nebulizable | |
| | | | organic | |
| | | | solvent | |
| Bovine serum | | Sigma | Aqueous | Modified BSA |
| albumin (BSA) | | Aldrich, | solution | such as, e.g. |
| | | St. | | biotinylated BS |
| | | Louis, | | or BS |
| | | MO | | conjugated to |
| | | | | small molecules |
| | | | | or haptens; other |
| | | | | proteins such as, |
| | | | | e.g., ovalbumin |
| Anti-rabbit IgG | | Sigma | Aqueous | Other antibodies |
| | | Aldrich, | solution | such as, e.g., |
| | | St. | | rabbit |
| | | Louis, | | immunoglobulin, |
| | | MO | | sheep |
| | | | | immunoglobulin, |
| | | | | anti-sheep |
| | | | | immunoglobulin; |
| | | | | immunoglobulin |
| | | | | class is not |
| | | | | critical and so |
| | | | | can use IgG, |
| | | | | IgA, IgM, etc.; |
| | | | | antibody |
| | | | | fragments, single |
| | | | | chain antibody |
| | | | | fragments |
| | | | | (scFvs), etc. |
| Poly-L-lysine | H₃N—CH(CH₂)₄NH₃Br—[CO—NH—CH(CH₂)₄NH₃Br]—COO | Sigma | Aqueous | Other |
| hydrobromide | | Aldrich, | solution | polycationic |
| | | St. | | polymers |
| | | Louis, | | comprising a |
| | | MO | | leaving group |
| Fluorescein | | Sigma | Aqueous | Other |
| isothiocyanate | | Aldrich, | solution | fluorescent dyes |
| (FITC) | | St. | | |
| | | Louis, | | |
| | | MO | | |

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

Methods

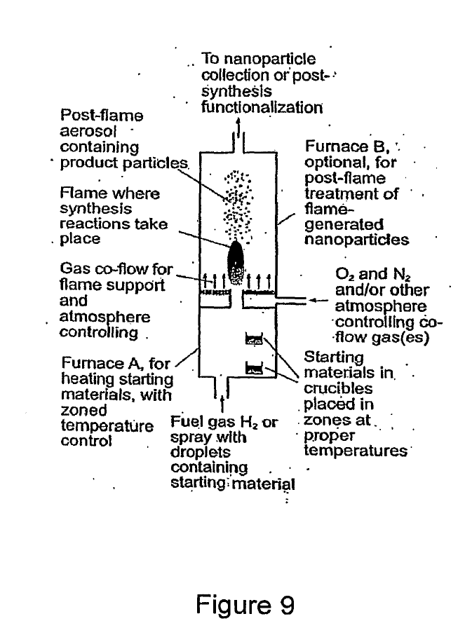

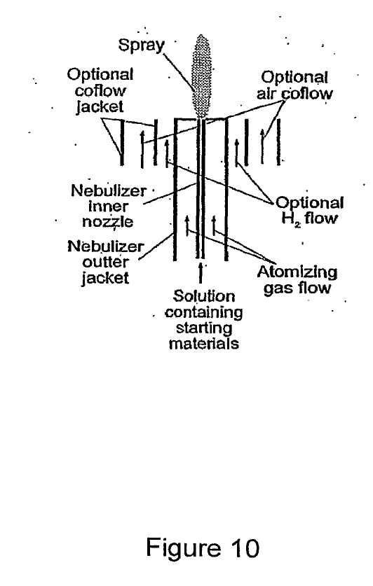

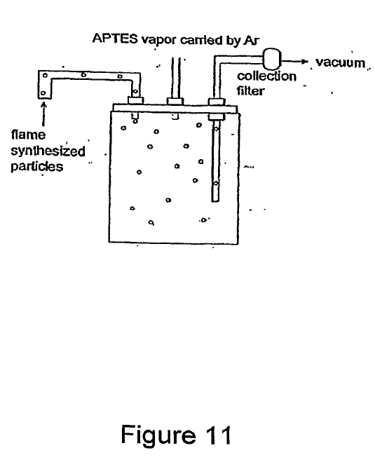

The syntheses of the particles useful in conjunction with the methods of the invention, as described herein, have been conducted in a manner that involves a flame as the reaction zone, utilizing an apparatus illustrated in FIG. 9 and FIG. 10, or a combination of the two. Functionalization has been carried out using the apparatus illustrated in FIG. 11, with an aerosol containing nanoparticles produced by the described syntheses as targets for functionalization.

Based on the different forms of usage of the starting materials, the syntheses can be divided into two classes, gas-phase synthesis in which all the starting materials are fed into the flame in the vapor phase, and, spray-pyrolysis synthesis in which one or more of the starting materials is fed into the flame in the form of droplets containing the starting material, or solid particles derived from the droplets. The functionalization methods of the present invention may be practiced with nanoparticles synthesized using the disclosed gas-phase combustion and/or pyrolysis synthesis method disclosed herein, or with nanoparticles produced using other manufacturing techniques.

### Example 1

### Gas-Phase Synthesis of Eu Nanoparticles

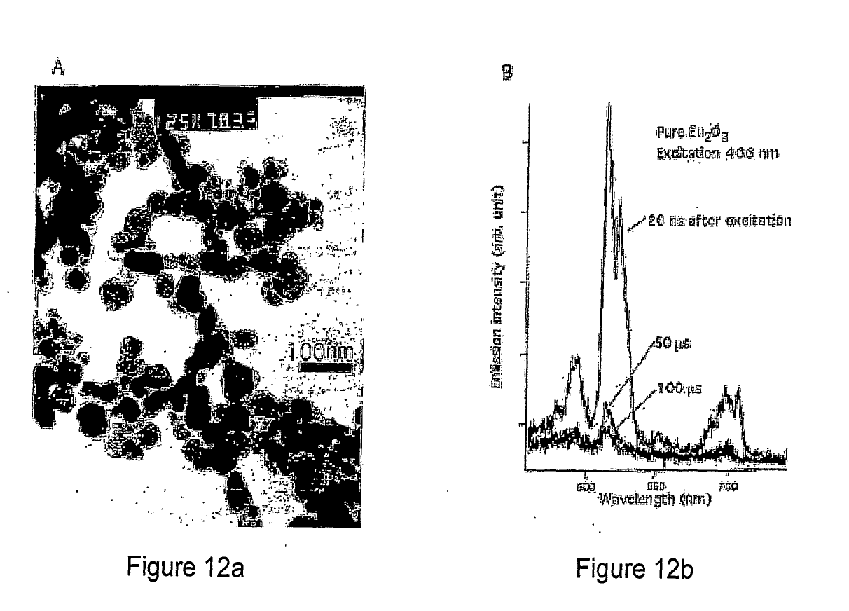

50 mg Eu(TMHD)₃ was placed in furnace A shown in FIG. 9. The material was heated to 200° C. and entrained in a stream of H₂ gas. The H₂ containing the starting materials was ignited at the outlet of furnace A in 1 atmosphere air. The maximum temperature in the flame was about 2130° C. The starting material decomposed in the flame, formed the corresponding oxide (i.e., Eu₂O₃). FIG. 12, left panel is a transmission electron micrograph of the material synthesized in this example, showing the size and morphology of the nanoparticles. Powder diffraction analysis revealed that the resulting crystals are monoclinic. Right panel of FIG. 12 is a fluorescence emission spectrum using an excitation wavelength of 466 nm. The fluorescence lifetime is short due to the small size of the nanoparticles and concentration quenching.

### Example 2

### Spray-Pyrolysis Synthesis of Eu:Y Nanoparticles

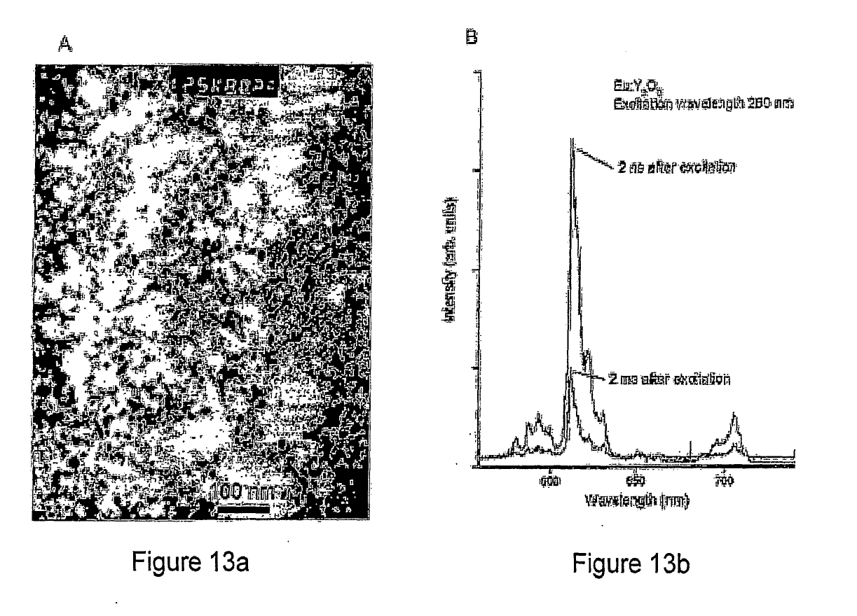

An ethanol solution containing 1 mM Eu(NO₃)₃ and 30 mM Y(NO₃)₃ was pumped with a syringe pump (Cole-Parmer, Vernon Hills, Ill.) at 7 mL/h into the inner nozzle of the nebulizer illustrated in FIG. 10. Ar gas, at 2 standard Liter/min, flowed through the annular gap surrounding the inner nozzle and atomized the ethanol solution containing the starting materials. The solution was atomized to form a spray at the tip of the nebulizer. The nebulizer was combined with an optional co-flow jacket, which supplied H₂ at 2 standard Liter/min and co-flowed air at 10 standard Liter/min, to form a hydrogen diffusion flame surrounding the outlet of the nebulizer. Flame temperature was about 2100° C. The H₂ diffusion flame ignited the spray formed by the nebulizer and reactions took place within the flame to form EU:Y₂O₃ nanoparticles that have desired chemical composition, size and morphology. FIG. 13 left panel shows a transmission electron micrograph of the resulting nanoparticles. The right panel of FIG. 13 shows a fluorescence emission spectrum using an excitation wavelength of 260 nm. Particles have a fluorescence lifetime on the order of 2 msec.

In an alternate method, the spray generated by the nebulizer can be introduced into furnace A, along with 2 standard Liter/min H₂. The spray then is preheated in furnace A to remove the solvent from the droplets, to form an aerosol containing dry particles. This aerosol can be ignited at the outlet of furnace A to form a diffusion flame, in which the synthesis reactions take place. Post-synthesis treatment of the nanoparticles produced by the spray-pyrolysis synthesis is optional with furnace B. Post-synthesis treatment helps to remove impurities and improve the crystallographic properties of the nanoparticles formed in the flame. In addition to ethanol, other solvents useful for spray pyrolysis include aqueous ethanol, water, acetone or other lower alcohols, ketones, or any other solvent in which the reagents are stable for the time necessary to carry out the synthesis, and that have a density and molecular weight appropriate to allow atomization of the reagents.

### Example 3

### Synthesis and Properties of the Magnetic Cores

Magnetic particles of Nd:Co:Fe₂O₃ mixed oxide were obtained by a spray pyrolysis method, previously reported for synthesis of Eu:Y₂O₃ nanoparticles [83]. Briefly, an ethanol solution containing Fe(NO₃)₃, Co(NO₃)₂ and Nd(NO₃)₃ was sprayed into a hydrogen diffusion flame through a nebulizer. The flame was formed by an H2 flow at 2 l min-1 and an air co-flow at 10 l min-1, surrounding the outlet of the nebulizer. A flame temperature of about 2000° C. was measured. Pyrolysis of the precursor solution within the flame yielded Nd:Co:Fe₂O₃ nanoparticles. A cold finger was used for collecting the particles thermophoretically. The production rate of this synthesis procedure was about 400-500 mg h-1. The ratio between Fe/Co/Nd salts was optimized experimentally to achieve the best magnetic characteristics.

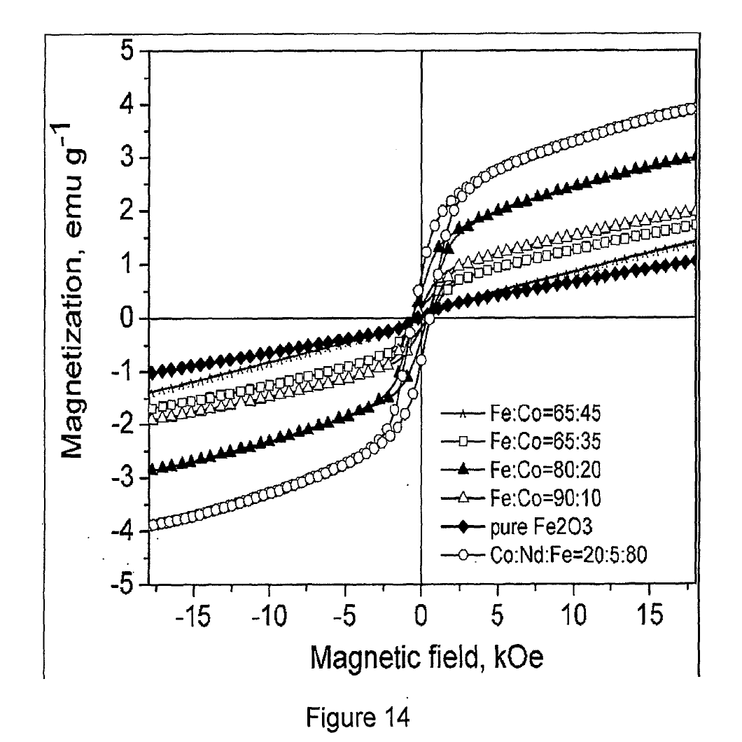

FIG. 14 shows the magnetic hysteresis loops of Co:Fe₂O₃ composite nanoparticles for powders obtained from liquid precursors with different mixing ratios of Co and Fe. The applied external magnetic field was in the range of 418 kOe. Pure Fe₂O₃ shows little magnetic hysteresis and small saturation magnetization. We attribute this to the high temperature during the synthesis process that does not favor the formation of magnetic γ-Fe₂O₃. However, adding Co(NO₃)₃ to the precursor mixture helped to improve the magnetic response, reaching maximum saturation magnetization with a Fe/Co ratio of 80/20. Further addition of Co to the precursor did not lead to improvement but to a decreasing of the saturation magnetization (see ratios Fe/Co of 65/35 and 65/45 in FIG. 14). Adding a small amount of Nd(NO₃)₃ to the precursor mixture helped to further increase the magnetization as shown in FIG. 14. The powder containing Nd reached about 25-30% higher saturation magnetization than the one without Nd. Adding larger amounts of Nd precursor did not improve the magnetization (data not shown).

### Example 4

### Synthesis and Properties of Core/Shell Magnetic Luminescent Particles

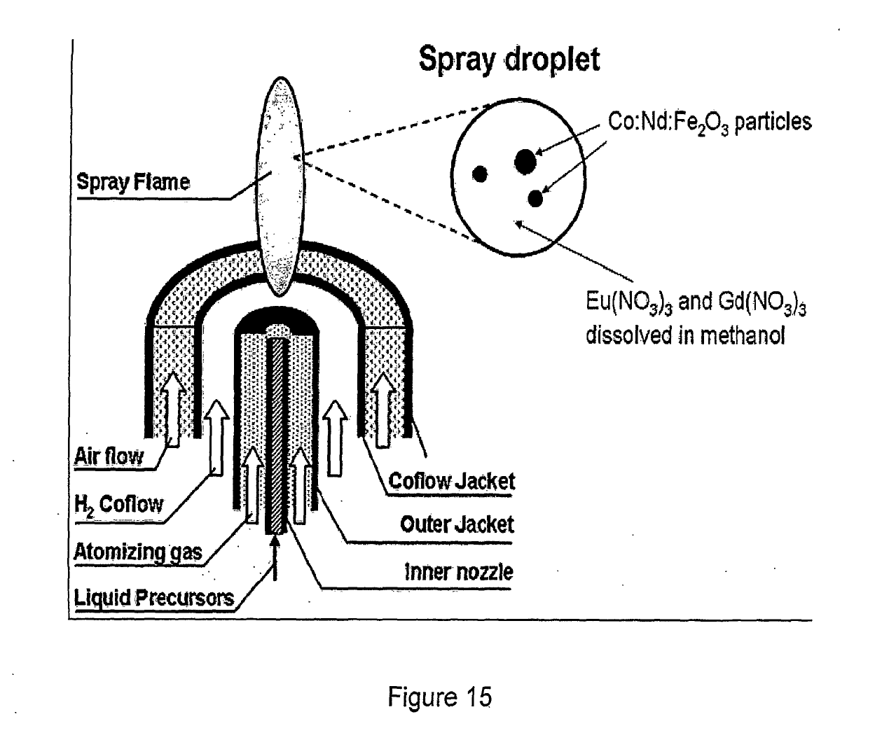

The Co:Nd:Fe₂O₃ powder with a Fe:Co:Nd ratio of 80:20:5 was used for the synthesis of core/shell particles, building the luminescent Eu:Gd₂O₃ shell via a second spray pyrolysis process. 10 mg of Co:Nd:Fe₂O₃ nanoparticles were dispersed in 50 ml ethanol containing 201mM Eu(NO₃)₃ and 80 mM Gd(NO₃)₃. The solution was subjected to an ultrasonic bath for 30 min in order to break any weak agglomerates. Afterwards it was sprayed through the hydrogen flame, as shown in FIG. 15. Gas and liquid flow rates were the same as described above. Each droplet of the formed spray contained solid magnetic particles and liquid precursors of Eu and Gd in ethanol. As a result, a composite particle containing magnetic cores and Eu:Gd₂O₃ luminescent shell was formed from each droplet in the flame. The Nd:Co:Fe₂O₃ nanoparticles in this case served as seeds for the formation of new particles in the spray. Other authors used seeds in a similar way for the synthesis of Eu:Y₂O₃ nanoparticles by spray pyrolysis [84].

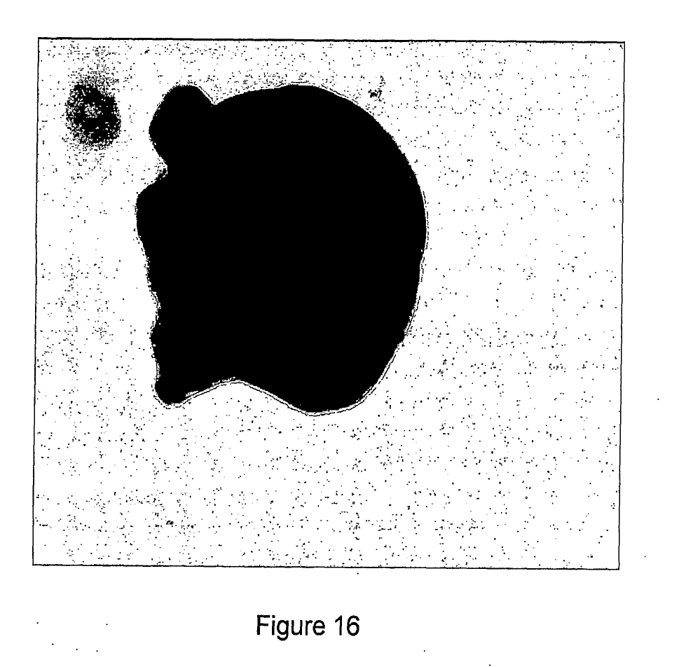

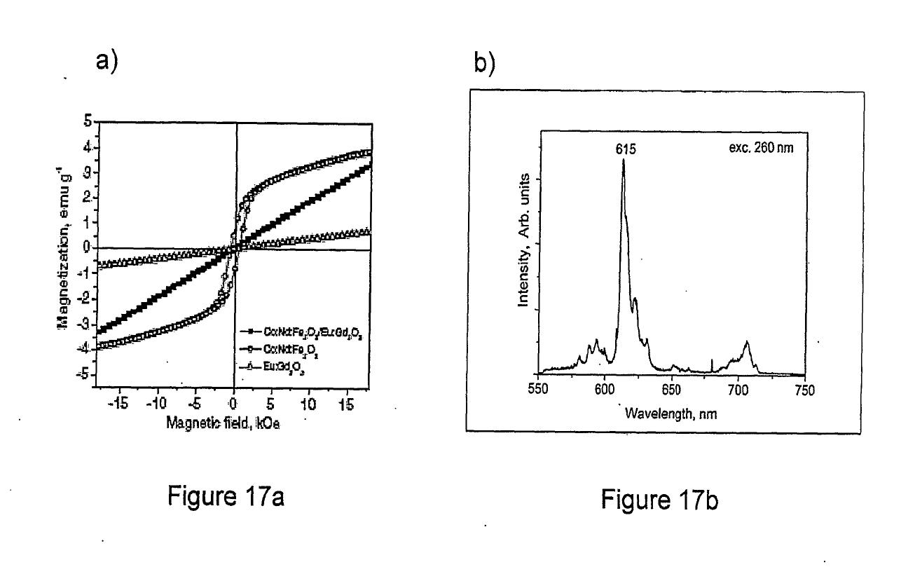

Analysis with a transmission electron microscope (TEM) revealed that most of the synthesized particles were in the size range between 200 nm and 1 μm depending on the number and the size of the embedded primary Co:Nd:Fe₂O₃ particles. A representative TEM image of a core/shell particle can be seen in FIG. 16. The image reveals the external shape of the particle as well as its internal morphology. The particle has an irregular form and an overall diameter of about 400 nm. Several primary Co:Nd:Fe₂O₃ particles of different sizes can be distinguished, all of them embedded in a shell with a thickness of 10-20 nm. The magnetic characteristics of the core (Co:Nd:Fe₂O₃) and the shell (Eu:Gd₂O₃) materials are compared to those of the final core/shell particles in FIG. 17a. While the cores exhibit ferromagnetic behavior, the core/shell particles after the second spray pyrolysis display a paramagnetic response. The aligned magnetization of the core/shell particles reaches nearly the same value as the ferromagnetic cores under an external magnetic field of ±18 kOe. In comparison, the magnetization of the shell material itself is much smaller. Note that in all cases the background contribution is negligible. This change of the magnetic characteristics from ferromagnetic core to paramagnetic core/shell particles can be attributed to a reduction or phase transformation of the magnetic core phase during the second spray pyrolysis process.

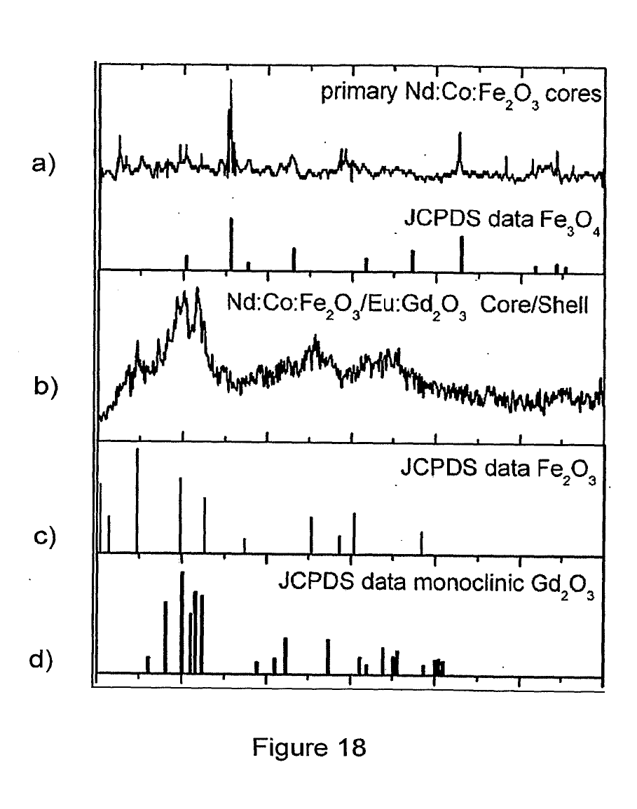

FIG. 17b shows the luminescent emission spectrum of the synthesized core/shell particles which is typical for the shell material Eu:Gd₂O₃. Under UV excitation at 260 nm, the particles with Eu:Gd₂O₃ shell emitted red luminescence with a narrow peak centered at 615 nm that was identical to the spectrum recently reported by the inventors for Eu:Gd₂O₃ nanoparticles [15]. The compatibility of Gd₂O₃ with the proposed synthesis process introduces the possibility for a variety of luminescent spectra to be achieved by using different lanthanides such as Th, Sm or Dy for doping [54]. X-ray diffraction studies have been performed on the primary obtained Nd:Co:Fe₂O₃ powder and the final core/shell particles in order to clarify the origin of the observed changes in the magnetic properties. The XRD spectrum of the primary magnetic particles reveal the presence of FeO₄ iron oxide along with other phases, as shown in FIG. 18a. The XRD spectrum of the secondary core/shell particles, shown in FIG. 18b, shows a pattern close to the monoclinic Gd₂O₃ phase, shown in FIG. 18d, which is in agreement with recently published results [53]. Some additional peaks (e.g. at 2θ=24.7°, 29.7°, 45.3°) coincide with strong peaks of Fe₂O₃, as shown in FIG. 18c. This leads to the conclusion that the core materials are changed (e.g. oxidized) during the second synthesis stage.

The final core/shell particles were successfully separated from an aqueous solution by a commercially available permanent magnet for biochemical purposes, as shown in FIG. 7. Initially, the particles that were suspended in water were attracted to the magnet and stuck to the glass wall, as shown in FIG. 7a. Afterwards, the water was pulled out of the tube leaving the particles in the tube, a shown in FIG. 7b. The attractive force that the particles experienced was sufficiently strong to prevent re-entrainment by the liquid flow during the removal of the water. This simple experiment demonstrates the applicability of the synthesized particles for separation purposes in biochemical protocols.

### Example 5

### Functionalization of Nanoparticles

Functionalization is carried out using the apparatus illustrated in FIG. 11. 4 ml of 3-aminopropyltriethoxy-silane (APTES) is contained in a 250 ml Erlenmeyer flask (not shown) having one inlet and one outlet, T=20° C., P=1 atm. Ar gas is used as a carrier gas to deliver APTES vapor into the reaction chamber of FIG. 1. Various flow rates of Ar are used: 50 SCCM, 75 SCCM, 100 SCCM, 150 SCCM.

The reaction chamber contains two inlets and one outlet. Nanoparticles are collected with a probe located 2-5 cm from the burner illustrated in FIG. 9. The flow rate of the combustion products gas into the chamber is determined by the vacuum suction rate. In the chamber, APTES vapor mixes with particles. The concentration of water in the aerosol plays an important role in the amino-silane coating of the target nanoparticles within. The presence of water molecule on the surface of the nanoparticles facilitates the binding of the amino-silane molecules with the particles surface. However, excess amounts of water cause cross-linking between the amino-silane molecules and render them useless or even detrimental to the coating process. Hence there is an optimal water vapor concentration for each functionalization process. In the case where nanoparticles are functionalized by coating with (3-Aminopropyl)triethoxysilane freshly from the gas-phase flame synthesis process, the water vapor is originated from the combustion of H₂ and its concentration in the aerosol is adjusted by dilution from the air co-flow assisting the combustion process. The water content in this aerosol is about 0.02 g/Liter, providing effective functionalization of these particles by APTES. The particle concentration in the aerosol is on the order of 106 particles/cm³, with a typical mean diameter of 50 nm. Functionalized particles are collected on the anodisc 47 Whatman filter.

### Example 6

### Biofunctionalization of the Core/Shell Particles

The magnetic luminescent nanoparticles were coated with antirabbit IgG via spontaneous physical adsorption according to a previously reported procedure for the biofunctionalization of Eu:Gd₂O₃ nanoparticles [7]. Briefly, a suspension of the nanoparticles in 25 mM phosphate buffer, pH=7, was incubated overnight in the antibody solution in a rotating mill at room temperature. The particles were extracted from the solution on a magnetic rack and washed three times. After that their surface was blocked with BSA to ensure that no bare particle surface remained.

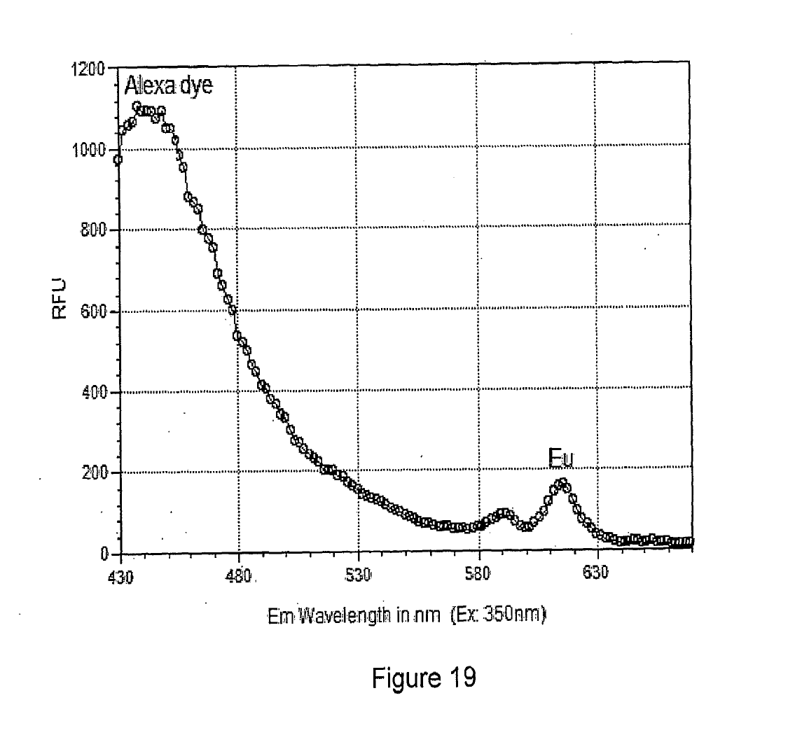

The concentrations of the coating antibody and labeled antigen were optimized in a titration experiment where 1 mg of particles, coated with anti-rabbit IgG in the concentration range of 10-500 μg mg-1 particles, were incubated for 1 h with different concentrations of rabbit IgG-Alexa Fluor 350. Negative controls were performed with magnetic nanoparticles coated with sheep IgG. After magnetic extraction, the nanoparticles were resuspended in 100 μl of PBS and the fluorescence of the resulting complex was measured on a Spectramax M2 microplate reader (Molecular Devices, Sunnyvale, Calif.). Both Eu:Gd2O3 and Alexa Fluor 350 were excited at 350 nm and their emission spectra were detected in the interval 430-670 nm. FIG. 19 represents the emission spectrum corresponding to 100 μg antibody/mg nanoparticles and 20 μg ml-1 rabbit IgG-Alexa Fluor 350. The intensity of Alexa Fluor 350 emission (at 445 nm) is proportional to the amount of labeled antigen bound to the particle surface while the intensity of Eu emission (at 615 nm) is related to the number of particles, and hence number of antibodies—the Eu signal serves as an internal standard.

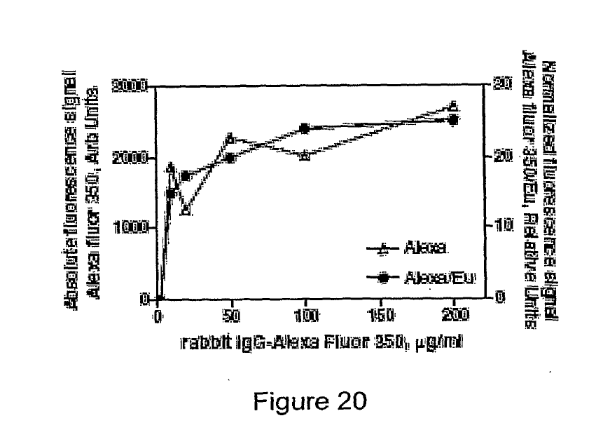

A typical titration curve representing the saturation of the immobilized antibody by the labeled antigen is presented in FIG. 20. The absolute measured signal of Alexa 350 is compared to the normalized signal (intensity ratio Alexa 350/Eu). Although the two curves show the same tendency toward saturation, using the internal fluorescent standard generates much smoother curves and more precise measurement. This approach eliminates the error due to possible variability in the magnetic particle extraction. The measured signal is relative instead of absolute, with the Eu signal as a measure for the amount of particles and antibodies that are interrogated in the plate reader. In this way, the intrinsic luminescence of the magnetic nanoparticles serves as an internal standard in the quantitative immunoassay. For the competitive magnetic immunoassay, the amount of coating antibody and the concentration of the labeled antigen were selected to generate a high signal-to-noise ratio. As a result of the titration experiments, we chose a coating antibody concentration of 100 μg antibody/mg nanoparticles and a concentration of rabbit IgG-Alexa Fluor 350 corresponding to 70% saturation of the capture antibody binding sites (about 20 μg ml-1). This internal standard procedure will facilitate the development and improvement of a variety of novel sensor formats where the separation and recovery of magnetic particles is not absolutely quantitative.

### Example 7

### Functionalization of Magnetic Microspheres

Tech data sheet #238E, Polysciences, Inc and Technote #204, Bangs Laboratories, Inc were used as protocols for direct physical adsorption of the antibodies on the magnetic particle surface. For IgG monolayer on the 1-2 μm diameter magnetic particles approximately 9.5 mg IgG/g beads are required [85]. It is recommended to use 3-10 times the amount of protein required for monolayer. Eight times the monolayer requirement (76 mg IgG/g beads) were used in this example. 50 μl of 2.52% polystyrene Superparamagnetic microspheres (1.26 mg particles) were separated from the solution using a magnetic rack and washed once with 1×PBS. Particles were mixed with required amount of antibodies (76 mg IgG/g beads≈48 μl of 2 mg/ml anti-mouse IgG-488 solution) and 3 ml of Borate buffer as the buffer solution. Solution was left for overnight (12 hrs) incubation at 4° C. After incubation magnetic particles were extracted from the solution using magnetic rack and supernatant was discarded. Particles were then washed once with Borate buffer (8.5pH) and stored in 2 ml of 0.1% BSA/PBS solution²⁴ (0.63 mg/ml solution).10 ml stock solution of 0.63 mg/ml of antibody coated magnetic particles was prepared for using above mentioned procedure.

### Example 8

### Competitive Immunoassay

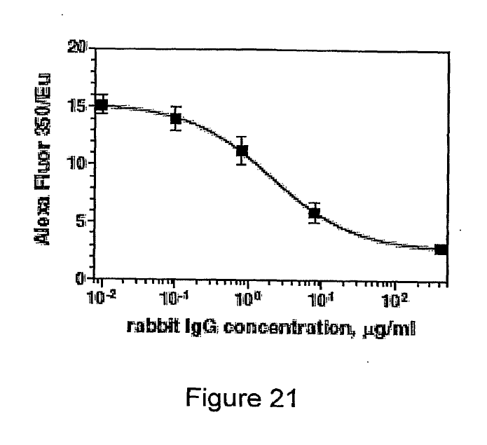

A competitive magnetic immunoassay for detection of rabbit IgG (target analyte) was performed on the functionalized particle surface. Half a mg of anti-rabbit IgG-coated magnetic nanoparticles was pre-incubated with the target analyte in 1 ml of 0.2% BSA/PBS for 1 h in a rotating mill at room temperature. After magnetic separation, the particles were incubated with a solution of 20 μg ml-1 of rabbit IgG-Alexa Fluor 350 for 1 h at room temperature. During this incubation the labeled IgG bound to the available binding sites on the particle surface. The amount of labeled antigen bound on the nanoparticle surface is inversely proportional to the amount of analyte in the sample during the first incubation. Finally the particles were extracted magnetically from the solution. In the detection step, the amount of bound, labeled antigen was quantified by the ratio between the intensities of Alexa 350 and Eu:Gd203. The measured rabbit IgG competitive curve is presented in FIG. 21. The parameters for the sigmoidal fit are as follows: IC50=2 μg ml⁻¹, slope=−0.75, R²=0.96. The LOD is ˜0.1 μg ml⁻¹. It is worth noticing the small standard deviations that emphasize the advantage of using an internal luminescent standard. Optimization of the assay sensitivity was not the subject of this work. Our main goal was to demonstrate that the novel luminescent magnetic nanoparticles can be successfully applied to assays based on magnetic separation and used as a substrate for the immobilization of biological receptors. This immunoassay method is potentially attractive for clinical applications in which magnetic separation is used.

### Example 9

### Conventional Assay Procedure

Different concentrations (˜0.2 to 25.6 μg/ml, 2× variation) of unlabeled mouse IgG were used as target antigen. The target antigen was diluted in 0.1% BSA/PBS solution and 100 μl of each concentration was incubated with 0.063 mg of anti-mouse IgG-488 coated magnetic particles in 12×75-mm borosilicate glass test tubes for 1 hr at 25° C. After incubation particles were extracted from the solution using magnetic rack and washed twice with 1×PBS solution. 100 μl of 15 μg/ml anti-mouse IgG-647 was then added to the particles and solution was incubated at 25° C. for 1 hr. Particles were again separated using magnetic rack, washed twice with 1×PBS solution and re-suspended in 100 μl 0.1% BSA/PBS. Solution was then poured into 96-well opaque microplate and fluorescence intensities were measured using Spectramax M2 cuvette/microplate spectrofluorometer with excitation at 485 nm, 620 nm and 260 nm.

For the kinetic studies only 0.2 μg/ml target antigen concentration was used. The time period of first incubation of primary antibody coated particles with target antigen was varied (˜0 to 180 mins, 10 min time step) and rest of the assay was performed as explained above.

### Example 10

### Conventional Sandwich Magnetic Immunoassay Using Anti-Mouse IgG-488 Coated Magnetic Particles

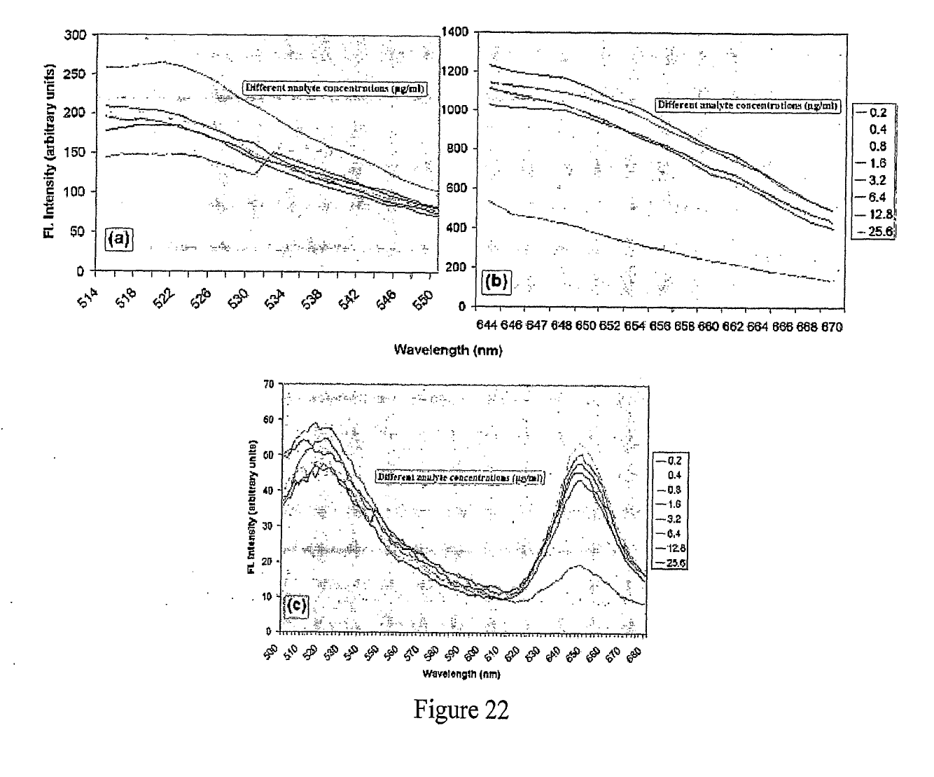

The organic dyes used for labeling the antibodies used in this experiment have a problem of spectral overlap due to small Stokes shift. To avoid spectral overlap in the spectrum author uses an excitation wavelength which is 10-20 nm lesser than the peak excitation wavelength of corresponding label. The emission spectrums of IgG-488 and IgG-635 after completion of the conventional immunoassay are presented in FIG. 22. Particles were excited at 480 nm (FIG. 22a), 620 nm (FIG. 22b) and 260 nm (FIG. 22c). The increase in fluorescence intensity (relative units) for IgG-635 label corresponds to increasing analyte concentration and consequent increase in number of IgG-635 labeled antibodies attached to the particle.

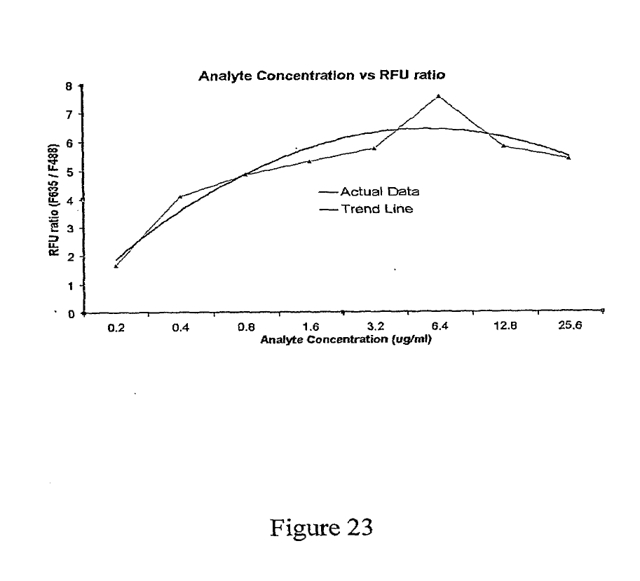

The peak emission wavelength values for IgG-635 and IgG-488 were used to create a standard base curve, as shown in FIG. 23, showing the fluorescence intensity ratio variation with changing analyte concentration. The number of antibody-antigen complexes will depend on the number of binding sites and the number of antibodies available. For a known concentration of uniformly antibody-coated magnetic particles there is definite number of binding sites available, excluding the effect of non-specific binding (0.1% BSA solution was used to inhibit non-specific analyte binding). After reaching the saturation, excessive analyte is washed away during washing steps. Therefore for a known concentration of antibody-coated magnetic particles there is a limit to bound analyte. This limit defines the maximum analyte concentration required for immuno-reaction kinetic studies. Base curve (FIG. 23) shows that the linear region for RFU ratio varying with changing analyte concentration extends till 0.8 μg/ml. Any concentration within the linear region would a good candidate for immuno-reaction kinetic studies.

Use of fluorescence intensity ratio to create the base curve eliminates the possible experimental error due the particle loss during washing steps. Fluorescence intensity ratio acts as internal standard unlike if the absolute value of fluorescence emission intensity from secondary label was to be used to create the curve.

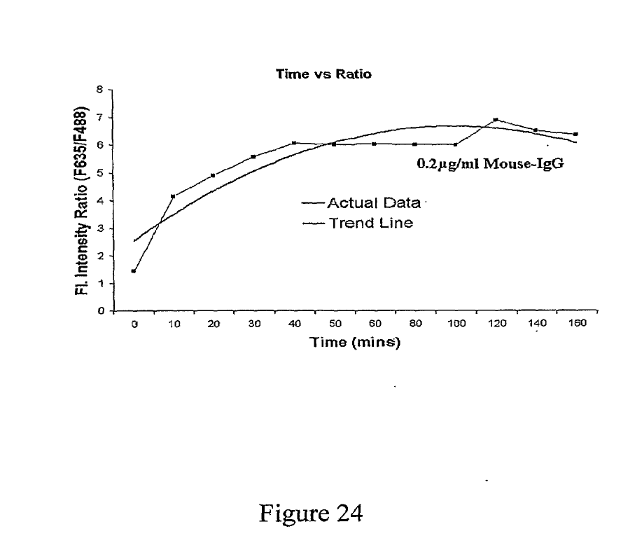

For immuno-reaction kinetic studies 10 mins time-step incubations were done for constant analyte concentration (0.2 μg/ml) and emission spectrum from each incubation were used to create another curve, as shown in FIG. 24, showing the time required to get to the saturation limit.

The linear variation of fluorescence intensity ratio with respect to time ends somewhere around 45-55 mins, which agrees with the standard protocol value.

### Example 11

### Assay in Microchannel

The same incubations as in Example 10 were performed inside the microchannel using known concentrations of magnetic particles and analytes. 31.5 μg (50 μl of stock solution) of IgG-635 coated magnetic particles were introduced in the channel. Particles were held against the channel wall by turning on one of the electromagnet and fluid was extracted from the channel using micro-pipet. 50 μl of analyte solution with known concentration (˜0.1 to 1.6 μg/ml, 2× variation) was then introduced in the channel. The electromagnets were turned on and off alternatively for 5 mins at 5 Hz switching frequency for mixing. After mixing the particles were again held against the channel wall and the channel was washed using 0.1% BSA solution. 50 μl solution of 15 μg/ml anti-mouse IgG-488 was then introduced in the channel and particles were again made to oscillate in the solution using the electromagnets. After mixing for 5 mins at 5 Hz switching frequency the second washing was done by holding the particles against the wall. Three samples were collected for each target antigen concentration and particles were re-suspended in 100 μl of 0.1% BSA/PBS solution. Solution was then poured into 96-well opaque microplate and fluorescence intensities were measured using Spectramax M2 cuvette/microplate spectrofluorometer with excitation at 260 nm.

### Example 12

### Sandwich Magnetic Immunoassay in Micro-Channel Using Anti-Mouse IgG-635 Coated Magnetic Particles

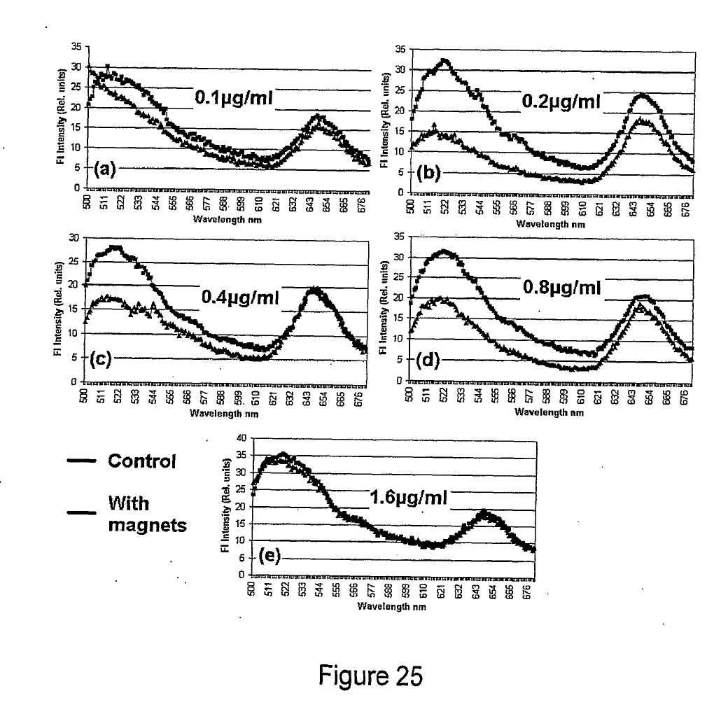

To investigate the effect of electromagnet augmented mixing on antigen-antibody reaction, inside the micro-channel, same incubations were done inside the channel without using electromagnets as control system. FIG. 25 shows immunoassays inside the channel with and without (control) using electromagnets for mixing, with different target antigen concentrations (a) 0.1 μg/ml (b) 0.2 μg/ml, (c) 0.4 μg/ml, (d) and (e) 1.6 μg/ml.

The foregoing description of the embodiments of the invention has been presented for the purposes of illustration and description. It is not intended to be exhaustive or to limit the invention to the precise forms disclosed. Persons skilled in the relevant art can appreciate that many modifications and variations are possible in light of the above teaching. Concentrations, sizes and other parameters stated in the specification and the claims are for example only and are intended to include variations consistent with the practice of the present invention. Such permissible variations are readily determined by persons of skill in the art in light of the instant disclosure and typically encompass between about +10% to about +20% of the stated parameter. It is therefore intended that the scope of the invention be limited not by this detailed description, but rather by the claims appended hereto. References to publications, patent applications and issued patents contained in this specification are herein incorporated by reference in their entirety for all purposes.

### REFERENCES

[1] Alivisatos P 2004 Nat. Biotechnol. 22 47-52
[2] Seydack M 2005 Biosensors Bioelectron. 20 2454-69
[3] Parak W J, Gerion D, Pellegrino T, Zanchet D, Micheel C, Williams S C, Boudreau R, Le Gros M A, Larabell C A and Alivisatos A P 2003 Nanotechnology 14 R15-27
[4] Jaiswal J K and Simon S M 2004 Trends Cell Biol. 14 497-504
[5] Tan M Q, Wang G L, Hai X D, Ye Z Q and Yuan J L 2004 J. Mater. Chem. 14 2896-901
[6] Harma H, Soukka T and Lovgren T 2001 Clin. Chem. 47 561-8
[7] Nichkova M, Dosev D, Perron R, Gee S J, Hammock B D and Kennedy I M 2006 Anal. Bioanal. Chem. 384 631-7
[8] Nichkova M, Dosev D, Gee S J, Hammock B D and Kennedy I M 2005 Anal. Chem. 77 6864-73
[9] Tan W H, Wang K M, He X X, Zhao X J, Drake T, Wang L and Bagwe R P 2004 Med. Res. Rev. 24 621-38
[10] Ye Z Q, Tan M Q, Wang G L and Yuan J L 2004 Anal. Chem. 76 513-8
Ye Z Q, Tan M Q, Wang G L and Yuan J L 2004 J. Mater. Chem. 14 851-6
[12] Huhtinen P, Soukka T, Lovgren T and Harma H 2004 J. Immun. Meth. 294 111-22
[13] Feng J, Shan G M, Maquieira A, Koivunen M E, Guo B, Hammock B D and Kennedy I M 2003 Anal. Chem. 75 5282-6
[14] Gerion D, Chen F Q, Kannan B, Fu A H, Parak W J, Chen D J, Majumdar A and Alivisatos A P 2003 Anal. Chem. 75 4766-72
[15] Dosev D, Nichkova M, Liu M Z, Guo B, Liu G Y, Hammock B D and Kennedy I M 2005 J. Biomed. Opt. 10 064006-064001/064007
[16] Cho S J, Kauzlarich S M, Olamit J, Liu K, Grandjean F, Rebbouh L and Long G J 2004 J. Appl. Phys. 95 6804-6
[17] Yoon T J, Kim J S, Kim B G, Yu K N, Cho M H and Lee J K 2005 Angew. Chem. Int. Edn 44 1068-71
[18] Wirix-Speetjens R and De Boeck J 2004 IEEE Trans. Magn. 40 1944-6
[19] Oswald P, Clement O, Chambon C, Schoumanclaeys E and Frija G 1997 Magn. Reson. Imag. 15 1025-31
[20] Kim D K, Zhang Y, Voit W, Kao K V, Kehr J, Bjelke B and Muhammed M 2001 Scr. Mater. 44 1713-7
Niemeyer C M 2001 Angew. Chem. Int. Edn 40 4128-58
[22] Zahn M 2001 J. Nanoparticle Res. 3 73-8
[23] Jain K K 2003 Expert Rev. Mol. Diagnostics. 3 153-61
[24] Hasan R, Bernard A, Ciccotelli J and Martin P 2003 Safety Sci. 41 155-79
[25] Lu Y, Yin Y D, Mayers B T and Xia Y N 2002 Nano Lett. 2 183-6
[26] Levy L, Sahoo Y, Kim K S, Bergey E J and Prasad P N 2002 Chem. Mater. 14 3715-21
[27] Sahoo Y, Goodarzi A, Swihart M T, Ohulchanskyy T Y, Kaur N, Furlani E P and Prasad P N 2005 J. Phys. Chem. B 109 3879-85
[28] Wang D S, He J B, Rosenzweig N and Rosenzweig Z 2004 Nano Lett. 4 409-13
[29] Mulvaney S P, Mattoussi H M and Whitman L J 2004 BioTechniques 36 602-9
[30] Lu H C, Yi G S, Zhao S Y, Chen D P, Guo L H and Cheng J 2004 J. Mater. Chem. 14 1336-41
[31] X. Zhao, L. Hilliard, S. Mechery, Y. Wang, R. Bagwe, S. Jin and W. Tan, “A rapid bioassay fox single bacterial cell quantitation using bioconjugated nanoparticles”, PNAS 10 1, pp. 15027-15032, 2004.
[32] Z. Muhammad-Tahir and E. C. Alocilja, “A disposable biosensor for pathogen detection in fresh produce samples”, Biosyst. Eng 88, pp. 145-151, 2004.
[33] E. T. Lagally, J. R. Scherer, R. G. Blazej, N. M. Toriello, B. A. Diep, M. Rarnchandani, G. F. Sensabaugh, L. W. Riley and R. A. Mathies, “Integrated portable genetic analysis microsystem for pathogenhnfectious disease-detection”, Anal Chem 76, pp. 3 162-3 170, 2004.
[34] T. Vo-Dinh, G. Griffin, I). L. Stokes, D. N. Stratis-Cullum, M. Askari and A. Wintenberg, “Multi-functional biochip for medical diagnostics and pathogen detection”, Optical Sensors Industrial, Environmental and Diagnostic Applications 1, pp. 12 1-143, 2004.
[35] K. Dill, C. Young and L. Stanker, “Detection of Salmonella in poultry using a silicon chip based biosensor”, J. Biochem. Biophys. Meth. 41, pp. 61-67, 1999.
[36] K. Sapsford, Y. Shubh, J. Delehanty, J. Golden, C. Taitt, L. Shriver-Lake and F. Ligler, “Fluorescence-based array biosensors for detection of biohazards”, J. Appl. Microbiol. 96, pp. 47-58, 2004.
[37] C. Taitt, J. Golden, Y. Shubin, L. Shriver-Lake, K. Sapsford, A. Rasooly and F. Ligler, “A portable array biosensor for detecting multiple analytes in complex samples”, Microb. Ecol. 47, pp. 175-185, 2004.
[38] B. Weeks, J. Camarero, A. Noy, A. Miller, L. Stanker and J. DeYoreo, “A microcantilever-based pathogen detector”, Scanning 25, pp. 2003.
[39] L. Beuchat, “Pathogenic microorganisms associated with fresh produce”, J. Food Protection 59, pp. 204-216, 1996.
[40] P. Mead, L. Slutsker, V. Dietz, L. McCaig, J. Bresee, C. Shapiro, P. Grifflin and R. Tauxe, “Food-related illness and death in the United States”, Emerg. Infect. Dis. 5, pp. 607-625, 1999.
[41] H. Moon, C. Kirk-Baer, M. Ascher, R. Cook, D. Franz, M. Hoy, D. Husnik, H. Jensen, K. Keller, J. Lederberg, M. L V, L. Powers, A. Steinberg, A. Strating, R. Smith, J. Kuzma, N. Grossblatt, L. Holliday, D. Sweatt and S. Strongin, “US agriculture is vulnerable to bioterrorism”, J. Vet. Med. Ed. 30, pp. 96-104, 2003.
[42] F. L. Mirarchi and M. Allswede, Ricin, http://www.emedicine.com/emerg/topic889.htm, 2004.
[43] S. Budavari, The Merck Index: an encyclopedia of chemicals, drugs, and biologicals, Merck and Co., Inc., Rahway N.J., 1989.
[44] S. Amon, R. Schechter, T. Inglesby, D. Henderson, J. Bartlett, M. Ascher, E. Eitzen, A. Fine, J. Hauer, M. Layton, S. Lillibridge, M. Osterholm, T. O'Toole, G. Parker, T. Perl, P. Russell, D. Swerdlow and K. Tonat, “Botulinum toxin as a biological weapon”, J. Am. Med. Assoc. 285, pp. 1059-1070, 2001.
[45] R. Wannemacher, J. Hewetson, P. Lemley, M. Poli, R. Dinterman, W. Thompson and D. Fram, Comparison of detection of ricin in castor bean extracts by bioassays, immunoassays, and chemistry procedures, in Recent Adv. Toxinol. Res, P. Gopalakrishnakone and C. Tan s.), Natl. Univ. Singapore Venom Toxin Res. Group, Singapore, pp. 108-119, 1992.
[46] M. Nogami and Y. Abe, “Properties of Sol-Gel-Derived Al203-Sio2 Glasses Using Eu3+ Ion Fluorescence Spectra”, J Non-Cryst Solids 197, pp. 73-78, 1996.
[47] A. Patra, E. Sominska, S. Ramesh, Y. Koltypin, Z. Zhong, H. Minti, R. Reisfeld and A. Gedanken, “Sonochemical preparation and characterization of Eu203 and Tb203 doped in and coated on silica and alumina nanoparticles”, Journal of Physical Chemistry B 103, pp. 3361-3365 1999.
[48] H. Harna, T. Soukka and T. Lovgren, “Europium nanoparticles and time-resolved fluorescence for ultrasensitive detection of prostate-specific antigen”, Clin Chem 47, pp. 561-568, 2001.
[49] J. Feng, G. M. Shan, A. Maquieira, M. E. Koivunen, B. Guo, B. D. Hammock and I. M. Kennedy, “Functionalized europium oxide nanoparticles used as a fluorescent label in an immunoassay for atrazine”, Anal Chem 75, pp. 5282-5286, 2003.
[50] D. Dosev, M. Nichkova, M. Liu, G. Liu, B. O. Hammock and I. M. Kennedy, “Application of fluorescent Eu:Gd203 nanoparticles to the visualization of protein micropatterns”, BIOS 2005, SPLE International Syrnp˜siump p. 2005.
[51] Montemayor S M, Garcia-Cerda L A and Torres-Lubian J R 2005 Mater. Lett. 59 1056-60
[52] Sinnecker E H C P, Gama S and Ribeiro C A 1991 J. Appl. Phys. 70 6480-2
[53] Goldys E M, Drozdowicz-Tomsia K, Jinjun S, Dosev D, Kennedy I M, Yatsunenko S and Godlewski M 2006 J. Am. Chem. Soc. 128 14498-505
[54] Gordon W O, Carter J A and Tissue B M 2004 J. Lumin. 108 339-42
[55] S. I. Tu, M. Golden, W. F. Fett, A. Gehring and P. Irwin, “Rapid detection of outbreak Escherichia coli 01 57 and Salmonella on alfalfa sprouts by immunomagnetic capture and time-resolved fluorescence”, J. Food Saf 23, pp. 75-89, 2003.
[56] S. P. Mulvaney, H. M. Mattoussi and L. J. Whitman, “Incorporating fluorescent dyes and quantum dots into magnetic microbeads for immunoassays”, Biotechniques 36, pp. 602-+, 2004.
[57] D. S. Wang, J. B. He, N. Rosenzweig and Z. Rosenzweig, “Superparamagnetic Fe203 Beads-CdSeIZnS quantum dots core-shell nanocomposite particles for cell separation”, Nano Letters 4, pp. 409-413, 2004.
[58] M. Qhobosheane, S. Swadeshmukul, P. Zhang and W. Tan, “Biochemically functionalized silica nanoparticles”, Analyst 126, pp. 1274-1278, 2001.
[59] Parak, W. J.; Gerion, D.; Pellegrino, T.; Zanchet, D.; Micheel, C.; Williams, S.C.; Boudreau, R.; Le Gros, M. A.; Larabell, C. A.; Alivisatos, A. P., Biological applications of colloidal nanocrystals. Nanotechnology 2003, 14, (7), R15-R27.
[60] Pankhurst, Q. A.; Connolly, J.; Jones, S. K.; Dobson, J., Applications of magnetic nanoparticles in biomedicine. Journal of Physics D-Applied Physics 2003, 36, (13), R167-R181.
[61] Bruchez, M.; Moronne, M.; Gin, P.; Weiss, S.; Alivisatos, A. P., Semiconductor nanocrystals as fluorescent biological labels. Science 1998, 281, (5385), 2013-2016.
[62] Chan, W. C. W.; Nie, S. M., Quantum dot bioconjugates for ultrasensitive nonisotopic detection. Science 1998, 281, (5385), 2016-2018.
[63] Han, M. Y.; Gao, X. H.; Su, J. Z.; Nie, S., Quantum-dot-tagged microbeads for multiplexed optical coding of biomolecules. Nature Biotechnology 2001, 19, (7), 631-635.
[64] Mah, C.; Fraites, T. J.; Zolotukhin, I.; Song, S. H.; Flotte, T. R.; Dobson, J.; Batich, C.; Byrne, B. J., Improved method of recombinant AAV2 delivery for systemic targeted gene therapy. Molecular Therapy 2002, 6, (1), 106-112.
[65] Edelstein, R. L.; Tamanaha, C. R.; Sheehan, P. E.; Miller, M. M.; Baselt, D. R.; Whitman, L. J.; Colton, R. J., The BARC biosensor applied to the detection of biological warfare agents. Biosensors & Bioelectronics 2000, 14, (10-11), 805-813.
[66] Nam, J. M.; Thaxton, C. S.; Mirkin, C. A., Nanoparticle-based bio-bar codes for the ultrasensitive detection of proteins. Science 2003, 301, (5641), 1884-1886.
[67] Mahtab, R.; Rogers, J. P.; Murphy, C. J., Protein-Sized Quantum-Dot Luminescence Can Distinguish between Straight, Bent, and Kinked Oligonucleotides. Journal of the American Chemical Society 1995, 117, (35), 9099-9100.
[68] D la Isla, A.; Brostow, W.; Bujard, B.; Esevez, M.; Rodriguez, J. R.; Vargas, S.; Castano, V. M., Nanohybrid scratch resistant coating for teeth and bone viscoelasticity manifested in tribology. Material Research Innovations 2003, 7, 110-114.
[69] Shinkai, M.; Yanase, M.; Suzuki, M.; Honda, H.; Wakabayashi, T.; Yoshida, J.; Kobayashi, T., Intracellular hyperthermia for cancer using magnetite cationic liposomes. Journal of Magnetism and Magnetic Materials 1999, 194, (1-3), 176-184.
[70] Molday, R. S.; MacKenzie, D., Immunospecific ferromagnetic iron dextran reagents for the labeling and magnetic separation of cells. Journal of Immunology Methods 1982, 52, 353-367.
[71] Weissleder, R.; Elizondo, G.; Wittenburg, J.; Rabito, C. A.; Begele, H. H.; Josephson, L., Ultrasmall superparamagnetic iron oxide: Characterization of a new class of contrast agents for MRI. Radiology 1990, 175, 489-493.
[72] Safarik, I.; Safarikova, M., Magnetic nanoparticles and biosciences. Monatshefte Fur Chemie 2002, 133, (6), 737-759.
[73] Widder, K. J.; Senyei, A. E.; Scarpelli, D. G., Magnetic microspheres: a model system for site specific drug delivery in vivo. Society for Experimental Biology and Medicine. Proceedings. 1978, 58, 141-146.
[74] Kriz, C. B.; Radevik, K.; Kriz, D., Magnetic permeability measurements in bioanalysis and biosensors. Analytical Chemistry 1996, 68, (11), 1966-1970.
[75] Richardson, J.; Hawkins, P.; Luxton, R., The use of coated paramagnetic particles as a physical label in a magneto-immunoassay. Biosensors & Bioelectronics 2001, 16, (9-12), 989-993.
[76] Richardson, J.; Hill, A.; Luxton, R.; Hawkins, P., A novel measuring system for the determination of paramagnetic particle labels for use in magneto-immunoassays. Biosensors & Bioelectronics 2001, 16, (9-12), 1127-1132.
[77] Enpuku, K.; Inoue, K.; Soejima, K.; Yoshinaga, K.; Kuma, H.; Hamasaki, N., Magnetic immunoassays utilizing magnetic markers and a high-T-C SQUID. Ieee Transactions on Applied Superconductivity 2005, 15, (2), 660-663.
[78] Enpuku, K.; Inoue, K.; Yoshinaga, K.; Tsukamoto, A.; Saitoh, K.; Tsukada, K.; Kandori, A.; Sugiura, Y.; Hamaoka, S.; Morita, H.; Kuma, H.; Hamasaki, N., Magnetic marker and high T-c superconducting quantum interference device for biological immunoassays. Ieice Transactions on Electronics 2005, E88C, (2), 158-167.
[79] Luxton, R.; Badesha, J.; Kiely, J.; Hawkins, P., Use of external magnetic fields to reduce reaction times in an immunoassay using micrometer-sized paramagnetic particles as labels (magnetoimmunoassay). Analytical Chemistry 2004, 76, (6), 1715-1719.
[80] Lee, G. U.; Metzger, S.; Natesan, M.; Yanavich, C.; Dufrene, Y. F., Implementation of force differentiation in the immunoassay. Analytical Biochemistry 2000, 287, (2), 261-271.
[81] Zborowski, M.; Fuh, C. B.; Green, R.; Sun, L. P.; Chalmers, J. J., Analytical Magnetapheresis of Ferritin-Labeled Lymphocytes. Analytical Chemistry 1995, 67, (20), 3702-3712.
[82] M. L. Chabinyc, D. T. Chiu, J. C. McDonald, A. D. Stroock, J. F. Christian, A. M. Karger and G. M. Whitesides, “An integrated fluorescence detection system in poly(dimethylsiloxane) for microfluidic applications”, Anal Chem 73, pp. 4491-4498, 2001.
[83] Dosev D, Guo B and Kennedy I M 2006 J. Aerosol Sci. 37 402-12
[84] Kang Y C, Roh H S and Park S B 2000 Adv. Mater. 12 451-3
[85] Bangs Laboratories, Inc., TechNote #204, Adsorption to Microspheres, Rev #001, 1999.

We claim:

1. An assay for determining the concentration of an analyte in a sample, comprising:
contacting the sample comprising the analyte with a luminescent magnetic particle under conditions in which the analyte specifically binds with the particle, wherein the particle is capable of light emission or absorption at a first wavelength, and a label capable of light emission or absorption at a second wavelength is associated with said analyte;
making a first measurement of the light emission or absorption at the first wavelength;
making a second measurement of the light emission or absorption at the second wavelength; and
calculating a ratio of the first and second measurements to determine the concentration of the analyte in the sample.

2. The method of claim 1, further comprising recording the determined analyte concentration.

3. The assay of claim 1, wherein no fluorescence resonance energy transfer occurs between the particle and the label.

4. The assay of claim 1, wherein the particle is a nanoparticle.

5. The assay of claim 4, wherein said nanoparticle comprises a magnetic core and a shell, said shell comprising one or more metal ions doped into a metal oxide host.

6. The assay of claim 5, wherein the nanoparticle further comprises a rare earth element doped in the metal oxide host.

7. The assay of claim 6, wherein the rare earth element is Europium.

8. The assay of claim 6, wherein the surface of said nanoparticle is functionalized with a biological molecule or a polyionic polymer.

9. The assay of claim 4, wherein the nanoparticle comprises a silica glass, and one or more metal ions doped into a metal oxide host.

10. The assay of claim 9, wherein the surface of said nanoparticle is functionalized with a biological molecule or a polyionic polymer.

11. A method of carrying out an assay on a magnetic particle disposed within a microchannel, comprising:
altering a position of a magnetic field within the microchannel; and
agitating a plurality of magnetic particles within the microchannel in response to the alteration of the magnetic field position, wherein the assay takes place on the surface of the magnetic particles.

12. The method of claim 11, wherein said altering is accomplished by alternatingly energizing a plurality of electromagnets.

13. The method of claim 11, further comprising:
stabilizing the position of the magnetic field to immobilize the plurality of magnetic particles within the microchannel.

14. The method of claim 11, further comprising:
exchanging a solution or obtaining a measurement while the plurality of magnetic particles are immobilized.

15. The method of claim 1, wherein the luminescent magnetic and the label are excited using a single excitation source.

16. The method of claim 11, wherein the plurality of magnetic particles are luminescent particles capable of light emission or absorption at a first wavelength.

17. The method of claim 11, wherein the assay is an immunoassay.

18. The method of claim 11, wherein the assay is the assay of claim 1.

19. The method of claim 11, wherein the plurality of particles are nanoparticles.

20. The method of claim 11, wherein the nanoparticles comprise a magnetic core and a shell, said shell comprising one or more metal ions doped into a metal oxide host.

21. The method of claim 20, wherein the nanoparticles further comprise a rare earth element doped in the metal oxide host.

22. The method of claim 21, wherein the rare earth element is Europium.
